(19) United States
(12) Patent Application Publication
(10) Pub. No.: US 2025/0043359 A1
(43) Pub. Date: Feb. 6, 2025

(54) ASSESSING AND TREATING MELANOMA

(71) Applicant: Mayo Foundation for Medical Education and Research, Rochester, MN (US)

(72) Inventor: Alexander Meves, Rochester, MN (US)

(21) Appl. No.: 18/717,252

(22) PCT Filed: Dec. 1, 2022
(86) PCT No.: PCT/US2022/051517

§ 371 Date: Jun. 6, 2024

Related U.S. Application Data
(60) Provisional application No. 63/287,217, filed on Dec. 8, 2021.

(51) Int. Cl. C12Q 1/6886; G01N 33/50; G01N 33/574

(52) U.S. Cl. CPC C12Q 1/6886; G01N 33/5091; G01N 33/5743; C12Q 2600/118; C12Q 2600/158; G01N 2800/52; G01N 2800/54

(57) ABSTRACT

This document relates to methods and materials for assessing and/or treating mammals (e.g., humans) having melanoma (e.g., primary cutaneous melanoma (PCM)). For example, methods and materials that can be used to determine whether or not a melanoma is likely to relapse (e.g., likely to relapse following treatment) are provided. For example, methods and materials for treating a mammal (e.g., a human) having melanoma where the treatment is selected based, at least in part, on whether or not the melanoma is likely to relapse are provided.

### CROSS REFERENCE TO RELATED APPLICATIONS

This application is a national phase entry under 35 U.S.C. § 371 of International Patent Application PCT/US2022/051517, filed Dec. 1, 2022, designating the United States of America and published as International Patent Publication WO 2023/107328 A1 on Jun. 15, 2023, which claims the benefit under Article 8 of the Patent Cooperation Treaty to U.S. Patent Application Ser. No. 63/287,217, filed Dec. 8, 2021. The disclosure of the prior application is considered part of (and is incorporated by reference in) the disclosure of this application.

### TECHNICAL FIELD

This document relates to methods and materials for assessing and/or treating mammals (e.g., humans) having melanoma (e.g., primary cutaneous melanoma (PCM)). For example, methods and materials provided herein can be used to determine whether or not a melanoma (e.g., a PCM) is likely to relapse (e.g., likely to relapse following treatment). This document also provides methods and materials for treating a mammal (e.g., a human) having melanoma (e.g., PCM) where the treatment is selected based, at least in part, on whether or not the melanoma is likely to relapse.

### BACKGROUND

PCM accounts for only 1-2% of all skin cancer diagnosed but approximately 75% of skin cancer deaths because of its extraordinary ability to metastasize. 5-year relative survival varies by stage and decreases with metastasis. Paradoxically, most relapses and deaths from PCM occur in individuals diagnosed with early-stage disease; i.e., patients with undetectable metastasis at diagnosis (stage I/II) and tumors of uncertain aggressiveness by conventional clinicopathologic staging parameters (Gershenwald et al., CA Cancer J. Clin., 67:472-92 (2017); and Morton et al., N. Engl. J. Med., 370:599-609 (2014)). The high number of deaths in early-stage patients is largely due to current inability to identify and treat high-risk early-stage disease. Stage III/IV patients (patients with metastasis) are eligible for adjuvant therapies and undergo regular surveillance imaging, and stage IIB-C patients are expected to become eligible for adjuvant treatment with pembrolizumab following the positive KEYNOTE-716 trial (Luke et al., Future Oncol., 16 (3): 4429-4438 (2020)). However, the large group of stage I/IIA patients are currently ineligible for adjuvant therapies.

### BRIEF SUMMARY

This document provides methods and materials for assessing and/or treating melanoma (e.g., PCM). In some cases, this document provides methods and materials for determining whether or not a melanoma (e.g., a PCM) is likely to relapse (e.g., likely to relapse following treatment). For example, a sample (e.g., a tissue sample containing one or more melanoma cells) obtained from a mammal having melanoma (e.g., PCM) can be assessed to determine if the melanoma is likely to relapse based, at least in part, on the presence or absence of altered levels (e.g., increased levels or decreased levels) of 4 or more (e.g., five, six, seven, eight, or nine) polypeptides (or mRNA encoding such polypeptides) in the sample. This document also provides methods and materials for treating a mammal (e.g., a human) having melanoma (e.g., PCM) where the treatment is selected based, at least in part, on whether or not the melanoma is likely to relapse. For example, a mammal (e.g., a human) having melanoma (e.g., PCM) can be administered one or more cancer treatments that are selected based, at least in part, on the presence or absence of altered levels (e.g., increased levels or decreased levels) of 4 or more polypeptides in a sample obtained from the mammal.

As demonstrated herein, the presence of an increased level of a holliday junction recognition protein (HJURP) polypeptide (or mRNA encoding a HJURP polypeptide), an increased level of a NUF2 component of NDC80 kinetochore complex (NUF2) polypeptide (or mRNA encoding a NUF2 polypeptide), an increased level of a protein phosphatase 1 regulatory subunit 9A (PPP1R9A) polypeptide (or mRNA encoding a PPPIR9A polypeptide), an increased level of a serine/arginine-rich splicing factor 11 (SRSF11) polypeptide (or mRNA encoding a SRSF11 polypeptide), an increased level of a 5′-nucleotidase domain-containing protein 3 (NT5DC3) polypeptide (or mRNA encoding a NT5DC3 polypeptide), an increased level of serpin family E member 2 (SERPINE2) polypeptide (or mRNA encoding a SERPINE2 polypeptide), a decreased level of a prostate androgen-regulated mucin-like protein 1 (PARM1) polypeptide (or mRNA encoding a PARM1 polypeptide), a decreased level of an epithelial membrane protein 2 (EMP2) polypeptide (or mRNA encoding an EMP2 polypeptide), a decreased level of a protein kinase C beta type (PRKCB) polypeptide (or mRNA encoding a PRKCB polypeptide), a decreased level of a T-cell surface glycoprotein CD1e (CD1E) polypeptide (or mRNA encoding a CD1E polypeptide), a decreased level of alcohol dehydrogenase 2 family member (ALDH2) polypeptide (or mRNA encoding an ALDH2 polypeptide), and a decreased level of CD200 receptor 1 (CD200R1) polypeptide (or mRNA encoding a CD200R1 polypeptide) in melanoma cells of a mammal having melanoma (e.g., PCM) can be used to identify that melanoma as being likely to relapse following treatment (e.g., likely to relapse following local/regional therapy).

Having the ability to identify whether a melanoma (e.g., PCM) is likely to relapse (e.g., likely to relapse following treatment) as described herein (e.g., based, at least in part, on the presence or absence of altered levels of 4 or more polypeptides in a sample obtained from a mammal having melanoma) provides a unique and unrealized opportunity to provide an individualized approach for selecting cancer therapies based on the risk of the patient thus providing cost-effective care with better outcomes. For example, a mammal having a high-risk melanoma (e.g., a melanoma that is identified as being likely to relapse as described herein) can be selected for adjuvant therapies, increased screening, and/or participation in clinical trials. For example, a mammal having a low-risk melanoma (e.g., a melanoma that is identified as not being likely to relapse as described herein) can forgo unnecessary treatment (e.g., lymph node surgery) and be spared from the toxicities and expenses.

In general, one aspect of this document features methods for assessing a mammal having a PCM. The methods can include, or consist essentially of, (a) determining if a sample from a mammal having PCM contains the presence or absence of (1) an increased level of a HJURP polypeptide or mRNA encoding the HJURP polypeptide, (2) an increased level of a NUF2 polypeptide or mRNA encoding the NUF2 polypeptide, (3) an increased level of a PPPIR9A polypeptide or mRNA encoding the PPPIR9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7) a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, or (12) a decreased level of a CD200R1 polypeptide or mRNA encoding the CD200R1 polypeptide, where the sample is a tissue sample comprising a melanoma cell; (b) classifying the PCM as being likely to relapse if the presence of at least 4 of (1)-(12) is determined; and (c) classifying the PCM as not being likely to relapse if the absence of each of (1)-(12) is determined. The mammal can be a human. The presence or absence of the increased level of the HJURP polypeptide or mRNA encoding the HJURP polypeptide can be determined. The presence or absence of the increased level of the NUF2 polypeptide or mRNA encoding the NUF2 polypeptide can be determined. The presence or absence of the increased level of the PPPIR9A polypeptide or mRNA encoding the PPPIR9A polypeptide can be determined. The presence or absence of the increased level of the SRSF11 polypeptide or mRNA encoding the SRSF11 polypeptide can be determined. The presence or absence of the increased level of the NT5DC3 polypeptide or mRNA encoding the NT5DC3 polypeptide can be determined. The presence or absence of the decreased level of the SERPINE2 polypeptide or mRNA encoding the SERPINE2 polypeptide can be determined. The presence or absence of the decreased level of the PARM1 polypeptide or mRNA encoding the PARM1 polypeptide can be determined. The presence or absence of the decreased level of the EMP2 polypeptide or mRNA encoding the EMP2 polypeptide can be determined. The presence or absence of the decreased level of the PRKCB polypeptide or mRNA encoding the PRKCB polypeptide can be determined. The presence or absence of the decreased level of the CD1E polypeptide or mRNA encoding the CD1E polypeptide can be determined. The presence or absence of the decreased level of the ALDH2 polypeptide or mRNA encoding the ALDH2 polypeptide can be determined. The presence or absence of the decreased level of the CD200R1 polypeptide or mRNA encoding the CD200R1 polypeptide can be determined. The method also can include determining a Breslow depth of the PCM. In some cases, the method can include determining the presence of the increased level of the HJURP polypeptide or mRNA encoding the HJURP polypeptide, the increased level of the NUF2 polypeptide or mRNA encoding the NUF2 polypeptide, the increased level of the PPPIR9A polypeptide or mRNA encoding the PPPIR9A polypeptide, the increased level of the SRSF11 polypeptide or mRNA encoding the SRSF11 polypeptide, the increased level of the NT5DC3 polypeptide or mRNA encoding the NT5DC3 polypeptide, the increased level of the SERPINE2 polypeptide or mRNA encoding the SERPINE2 polypeptide, the decreased level of the PARM1 polypeptide or mRNA encoding the PARM1 polypeptide, the decreased level of the EMP2 polypeptide or mRNA encoding the EMP2 polypeptide, the decreased level of the PRKCB polypeptide or mRNA encoding the PRKCB polypeptide, and the decreased level of the CD1E polypeptide or mRNA encoding the CD1E polypeptide; and where the Breslow depth can be from about 0.8 mm to about 10 mm, and can include classifying the PCM as being likely to relapse. In some cases, the method can include determining the absence of the increased level of the HJURP polypeptide or mRNA encoding the HJURP polypeptide, the increased level of the NUF2 polypeptide or mRNA encoding the NUF2 polypeptide, the increased level of the PPPIR9A polypeptide or mRNA encoding the PPPIR9A polypeptide, the increased level of the SRSF11 polypeptide or mRNA encoding the SRSF11 polypeptide, the increased level of the NT5DC3 polypeptide or mRNA encoding the NT5DC3 polypeptide, the increased level of the SERPINE2 polypeptide or mRNA encoding the SERPINE2 polypeptide, the decreased level of the PARM1 polypeptide or mRNA encoding the PARM1 polypeptide, the decreased level of the EMP2 polypeptide or mRNA encoding the EMP2 polypeptide, the decreased level of the PRKCB polypeptide or mRNA encoding the PRKCB polypeptide, and the decreased level of the CD1E polypeptide or mRNA encoding the CD1E polypeptide; and where the Breslow depth can be from about 0.1 mm to about 0.8 mm, and can include classifying the PCM as not being likely to relapse.

In another aspect, this document features methods for selecting a treatment for a mammal having a PCM. The methods can include, or consist essentially of, (a) determining that a sample from a mammal having a PCM contains the presence of at least 4 of: (1) an increased level of a HJURP polypeptide or mRNA encoding the HJURP polypeptide, (2) an increased level of a NUF2 polypeptide or mRNA encoding the NUF2 polypeptide, (3) an increased level of a PPPIR9A polypeptide or mRNA encoding the PPPIR9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide, where the sample is a tissue sample comprising a melanoma cell; and (b) selecting the mammal for surgery to remove the PCM and to receive an adjuvant therapy. The method also can include determining that the PCM has a Breslow depth of from about 0.8 mm to about 10 mm. The mammal can be a human. The adjuvant therapy can include radiation therapy. The adjuvant therapy can include administering an anti-cancer drug to the mammal. The anti-cancer drug can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for selecting a treatment for a mammal having a PCM. The methods can include, or consist essentially of, (a) determining that a sample from a mammal having a PCM contains the absence of: (1) an increased level of a HJURP polypeptide or mRNA encoding the HJURP polypeptide, (2) an increased level of a NUF2 polypeptide or mRNA encoding the NUF2 polypeptide, (3) an increased level of a PPPIR9A polypeptide or mRNA encoding the PPPIR9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide, where the sample is a tissue sample comprising a melanoma cell; and (b) selecting the mammal for surgery to remove the PCM in the absence of an adjuvant therapy. The method also can include determining that the PCM has a Breslow depth of from about 0.1 mm to about 0.8 mm. The mammal can be a human. The adjuvant therapy can include radiation therapy. The adjuvant therapy can include administering an anti-cancer drug to the mammal. The anti-cancer drug can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for treating a mammal having a PCM. The methods can include, or consist essentially of, (a) determining that a sample from a mammal having a PCM contains the presence of at least 4 of: (1) an increased level of a HJURP polypeptide or mRNA encoding the HJURP polypeptide, (2) an increased level of a NUF2 polypeptide or mRNA encoding the NUF2 polypeptide, (3) an increased level of a PPPIR9A polypeptide or mRNA encoding the PPPIR9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide, where the sample is a tissue sample comprising a melanoma cell; and (b) subjecting the mammal to a surgery to remove the PCM; and (c) administering an adjuvant therapy to the mammal. The method also can include determining that the PCM has a Breslow depth of from about 0.8 mm to about 10 mm. The mammal can be a human. The adjuvant therapy can include radiation therapy. The adjuvant therapy can include administering an anti-cancer drug to the mammal. The anti-cancer drug can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for treating a mammal having a PCM. The methods can include, or consist essentially of, subjecting a mammal having a PCM to a surgery to remove the PCM and administering an adjuvant therapy to the mammal, when the mammal is identified as having at least 4 of: (1) an increased level of a HJURP polypeptide or mRNA encoding the HJURP polypeptide, (2) an increased level of a NUF2 polypeptide or mRNA encoding the NUF2 polypeptide, (3) an increased level of a PPPIR9A polypeptide or mRNA encoding the PPPIR9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide in a sample obtained from the mammal, where the sample is a tissue sample comprising a melanoma cell. The method also can include determining that the PCM has a Breslow depth of from about 0.8 mm to about 10 mm. The mammal can be a human. The adjuvant therapy can include radiation therapy. The adjuvant therapy can include administering an anti-cancer drug to the mammal. The anti-cancer drug can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for treating a mammal having a PCM. The methods can include, or consist essentially of, (a) determining if a sample from a mammal having a PCM contains the absence of (1) an increased level of a HJURP polypeptide or mRNA encoding the HJURP polypeptide, (2) an increased level of a NUF2 polypeptide or mRNA encoding the NUF2 polypeptide, (3) an increased level of a PPPIR9A polypeptide or mRNA encoding the PPPIR9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide, where the sample is a tissue sample comprising a melanoma cell; and (b) subjecting the mammal to a surgery to remove the PCM in the absence of an adjuvant therapy. The method also can include determining that the PCM has a Breslow depth of from about 0.1 mm to about 0.8 mm. The mammal can be a human. The adjuvant therapy can include radiation therapy. The adjuvant therapy can include administering an anti-cancer drug to the mammal. The anti-cancer drug can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for treating a mammal having a PCM. The methods can include, or consist essentially of, subjecting a mammal having a PCM to a surgery to remove the PCM in the absence of an adjuvant therapy, when the mammal is identified as lacking: (1) an increased level of a HJURP polypeptide or mRNA encoding the HJURP polypeptide, (2) an increased level of a NUF2 polypeptide or mRNA encoding the NUF2 polypeptide, (3) an increased level of a PPPIR9A polypeptide or mRNA encoding the PPPIR9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide in a sample obtained from the mammal, where the sample is a tissue sample comprising a melanoma cell. The method also can include determining that the PCM has a Breslow depth of from about 0.1 mm to about 0.8 mm. The mammal can be a human. The adjuvant therapy can include radiation therapy. The adjuvant therapy can include administering an anti-cancer drug to the mammal. The anti-cancer drug can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

Unless otherwise defined, all technical and scientific terms used herein have the same meaning as commonly understood by one of ordinary skill in the art to which this disclosure pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the invention will be apparent from the description and drawings, and from the claims.

### BRIEF DESCRIPTION OF THE DRAWINGS

FIG. 1 shows an exemplary method for step-by-step filtering of genes derived from RNA sequencing experiments to identify markers for predicting melanoma relapse.

FIG. 2 contains graphs showing receiver operating characteristics (ROCs) from LASSO survival models at different time points. The models are derived from normalized gene expression data obtained by RNA sequencing experiments.

FIG. 3 contains a graph showing variables that appear at least 10% of times in the 300 models. These variables are derived from data obtained by RNA sequencing experiments.

FIG. 4 contains a table showing exemplary polypeptides having altered levels in melanoma cells from PCMs that are likely to relapse. These results are derived from data obtained by RNA sequencing experiments.

FIG. 5 contains graphs showing receiver operating characteristics (ROCs) from LASSO survival models at different time points. The models are derived from normalized gene expression data obtained by quantitative PCR experiments.

FIG. 6 contains a graph showing variables that appear at least 10% of times in the 300 models. These variables are derived from data obtained by quantitative PCR experiments.

FIG. 7 contains a table showing exemplary polypeptides having altered levels in melanoma cells from PCMs that are likely to relapse. These results are derived from quantitative PCR experiments.

FIG. 8 shows levels of a HJURP polypeptide and mRNA encoding a HJURP polypeptide in humans.

FIG. 9 shows levels of a NUF2 polypeptide and mRNA encoding a NUF2 polypeptide in humans.

FIG. 10 shows levels of a PPPIR9A polypeptide and mRNA encoding a PPP1R9A polypeptide in humans.

FIG. 11 shows levels of a SRSF11 polypeptide and mRNA encoding a SRSF11 polypeptide in humans.

FIG. 12 shows levels of a NT5DC3 polypeptide and mRNA encoding a NT5DC3 polypeptide in humans.

FIG. 13 shows levels of a SERPINE2 polypeptide and mRNA encoding a SERPINE2 polypeptide in humans.

FIG. 14 shows levels of a PARM1 polypeptide and mRNA encoding a PARM1 polypeptide in humans.

FIG. 15 shows levels of an EMP2 polypeptide and mRNA encoding an EMP2 polypeptide in humans.

FIG. 16 shows levels of a PRKCB polypeptide and mRNA encoding a PRKCB polypeptide in humans.

FIG. 17 shows levels of a CD1E polypeptide and mRNA encoding a CD1E polypeptide in humans.

FIG. 18 shows levels of an ALDH2 polypeptide and mRNA encoding an ALDH2 polypeptide in humans.

FIG. 19 shows levels of a CD200R1 polypeptide and mRNA encoding a CD200R1 polypeptide in humans.

### DETAILED DESCRIPTION

This document provides methods and materials that can be used to determine whether or not a melanoma (e.g., a PCM) is likely to relapse (e.g., likely to relapse following treatment). For example, a sample (e.g., a tissue sample containing one or more melanoma cells) obtained from a mammal having melanoma (e.g., PCM) can be assessed for the presence or absence of an altered level (e.g., an increased level or a decreased level) of 4 or more polypeptides in the sample to determine whether or not the melanoma is likely to relapse. In some cases, the methods and materials provided herein also can include treating a mammal having melanoma (e.g., PCM) where one or more cancer treatments are selected based, at least in part, on whether or not the melanoma is likely to relapse.

A mammal (e.g., a human) having melanoma (e.g., PCM) can be assessed to determine whether or not the melanoma is likely to relapse (e.g., likely to relapse following treatment) by detecting the presence or absence of an altered level (e.g., an increased level or a decreased level) of 4 or more polypeptides in a sample (e.g., a tissue sample containing one or more melanoma cells) obtained from the mammal. As described herein, the presence or absence of an altered level (e.g., an increased level or a decreased level) of 4 or more polypeptides in a sample (e.g., a tissue sample containing one or more melanoma cells) obtained from a mammal having melanoma (e.g., PCM) can be used to determine whether or not that melanoma is likely to relapse.

Any appropriate mammal having melanoma (e.g., PCM) can be assessed and/or treated as described herein. Examples of mammals that can have melanoma (e.g., PCM) and can be assessed and/or treated as described herein include, without limitation, humans, non-human primates (e.g., monkeys), dogs, cats, horses, cows, pigs, sheep, mice, and rats. In some cases, a human having melanoma (e.g., PCM) can be assessed and/or treated as described herein.

When assessing and/or treating a mammal (e.g., a human) having melanoma as described herein, the melanoma can be any type of melanoma. A melanoma can be any stage of melanoma (e.g., stage 0, stage I, stage II, stage III, or stage IV). A melanoma can be at any location on a mammal (e.g., a human) being assessed and/or treated as described herein. In some cases, a melanoma can be at a location on a mammal's body that has been exposed to ultraviolet (UV) radiation. For example, a melanoma can be at a location on a mammal's body that has had exposure to the sun. For example, a melanoma can be at a location on a mammal's body that has had exposure to a tanning lamp and/or a tanning bed. Examples of locations on a mammal's body where a melanoma that can be assessed and/or treated as described herein include, without limitation, chest, back, legs, arms, face, soles of the feet, palms of the hands, fingernail beds, and/or toenail beds.

In some cases, the methods described herein can include identifying a mammal (e.g., a human) as having melanoma (e.g., PCM). Any appropriate method can be used to identify a mammal as having melanoma (e.g., PCM). For example, physical examination (e.g., to examine the skin to look for signs that may indicate melanoma), imaging techniques (e.g., X-rays, computerized tomography (CT) scanning, and positron emission tomography (PET)), laboratory examination (e.g., of a skin sample obtained by biopsy), and/or laboratory tests (e.g., blood tests for elevated lactate dehydrogenase (LDH), the presence of a BRAFV600E mutation, and the presence of DNA methylation markers) can be used to identify a mammal (e.g., a human) as having melanoma (e.g., PCM). In some cases, a mammal can be identified as having melanoma (e.g., PCM) without having been subjected to a sentinel lymph node biopsy (SLNb). In some cases, a mammal can be identified as having melanoma (e.g., PCM) and having a negative SLNb.

In some cases, a melanoma (e.g., PCM) can be identified as likely to relapse (e.g., likely to relapse following treatment) based, at least in part, on the presence of an altered level of 4 or more polypeptides (or mRNA encoding such polypeptides) in a sample (e.g., a tissue sample containing one or more melanoma cells) obtained from a mammal having the melanoma. In some cases, an altered level can be an increased level of one or more polypeptides (or mRNA encoding such polypeptides) in a sample (e.g., a tissue sample containing one or more melanoma cells) obtained from a mammal having melanoma (e.g., PCM). The term “increased level” as used herein with respect to a level of a polypeptide (or mRNA encoding that polypeptide) in a sample refers to any level that is higher than a reference level of the polypeptide (or mRNA). In some cases, an altered level can be a decreased level of a polypeptide (or mRNA encoding that polypeptide) in a sample (e.g., a tissue sample containing one or more melanoma cells) obtained from a mammal having melanoma (e.g., PCM). The term “decreased level” as used herein with respect to a level of a polypeptide (or mRNA encoding that polypeptide) in a sample refers to any level that is lower than a reference level of the polypeptide (or mRNA). The term “reference level” as used herein with respect to a level of a polypeptide (or mRNA) refers to the level of the polypeptide (or mRNA) typically observed in a control sample. Control samples are samples obtained from mammals having melanoma at the time of initial diagnosis and having a SLNb performed within 90 days of the diagnosis, where the mammal after treatment experienced relapse-free survival for at least 4 years. It will be appreciated that levels of polypeptides (or mRNAs) from comparable samples are used when determining whether or not a particular level is an altered level of a polypeptide (or mRNA).

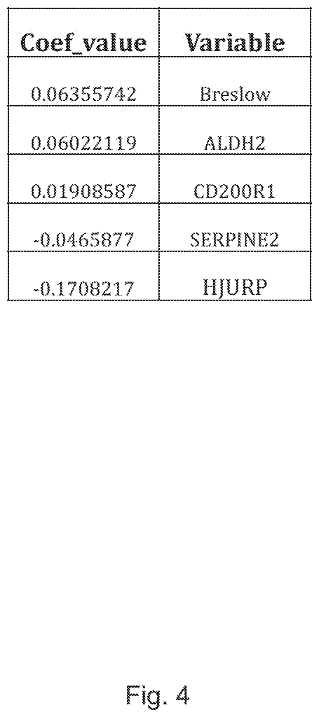

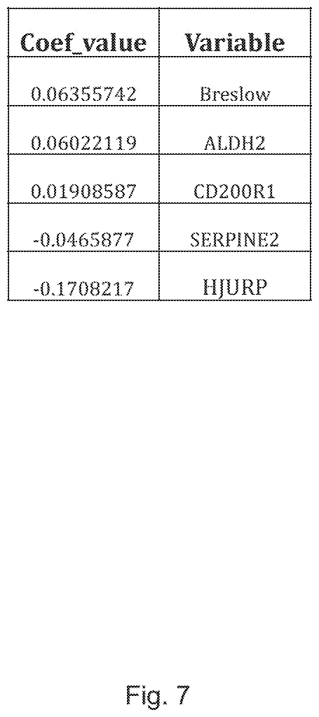

A melanoma (e.g., PCM) that is likely to relapse (e.g., likely to relapse following treatment) can have the presence of an altered level of any appropriate polypeptides (or mRNAs encoding the polypeptides) in a sample (e.g., a tissue sample containing one or more melanoma cells) obtained from a mammal having the melanoma. Examples of polypeptides that can be assessed to determine if an elevated level is present or absent within a sample (e.g., a tissue sample containing one or more melanoma cells) obtained from a mammal having melanoma (e.g., PCM) as described herein include, without limitation, a HJURP polypeptide, a NUF2 polypeptide, a PPP1R9A polypeptide, a SRSF11 polypeptide, a NT5DC3 polypeptide, a PARM1 polypeptide, an EMP2 polypeptide, a PRKCB polypeptide, a CD1E polypeptide, an ALDH2 polypeptide, a CD200R1 polypeptide, a SERPINE2 polypeptide, and a SPP1 polypeptide. Examples of mRNA that can be assessed to determine if an altered level is present or absent within a sample (e.g., a tissue sample containing one or more melanoma cells) obtained from a mammal having melanoma (e.g., PCM) as described herein include, without limitation, mRNAs encoding a HJURP polypeptide, a NUF2 polypeptide, a PPPIR9A polypeptide, a SRSF11 polypeptide, a NT5DC3 polypeptide, a PARM1 polypeptide, an EMP2 polypeptide, a PRKCB polypeptide, a CD1E polypeptide, an ALDH2 polypeptide, a CD200R1 polypeptide, a SERPINE2 polypeptide, or a SPP1 polypeptide. In some cases, a polypeptide that can be assessed to determine if an elevated level is present or absent within a sample (e.g., a tissue sample containing one or more melanoma cells) obtained from a mammal having melanoma (e.g., PCM) can be as listed in FIG. 4 or FIG. 7. In some cases, an mRNA encoding a polypeptide as listed in FIG. 4 or FIG. 7 can be assessed to determine if an altered level is present or absent within a sample (e.g., a tissue sample containing one or more melanoma cells) obtained from a mammal having melanoma (e.g., PCM).

In some cases, a melanoma (e.g., PCM) is not assessed for an altered (e.g., an increased or a decreased) level of a TGFBR1 polypeptide (and an mRNA encoding a TGFBR1 polypeptide), a SERPINE2 polypeptide (and an mRNA encoding a SERPINE2 polypeptide), a CXCL8 polypeptide (and an mRNA encoding a CXCL8 polypeptide), a GDF15 polypeptide (and an mRNA encoding a GDF15 polypeptide), a PLAT polypeptide (and an mRNA encoding a PLAT polypeptide), a MLANA polypeptide (and an mRNA encoding a MLANA polypeptide), a ITGB3 polypeptide (and an mRNA encoding a ITGB3 polypeptide), and/or a LOXL4 polypeptide (and an mRNA encoding a LOXL4 polypeptide).

In some cases, a melanoma (e.g., PCM) that is likely to relapse (e.g., likely to relapse following treatment) can lack an increased level of a TGFBR1 polypeptide (or an mRNA encoding a TGFBR1 polypeptide), an increased level of a SERPINE2 polypeptide (or an mRNA encoding a SERPINE2 polypeptide), an increased level of a CXCL8 polypeptide (or an mRNA encoding a CXCL8 polypeptide), an increased level of a GDF15 polypeptide (or an mRNA encoding a GDF15 polypeptide), an increased level of a PLAT polypeptide (or an mRNA encoding a PLAT polypeptide), an increased level of a MLANA polypeptide (or an mRNA encoding a MLANA polypeptide), an increased level of a ITGB3 polypeptide (or an mRNA encoding a ITGB3 polypeptide), and/or an increased level of a LOXL4 polypeptide (or an mRNA encoding a LOXL4 polypeptide).

In some cases, an altered level of a polypeptide (or mRNA) can be an increased level of a HJURP polypeptide (or an increased level of an mRNA encoding a HJURP polypeptide). Examples of HJURP polypeptides and nucleic acid sequences encoding a HJURP polypeptide include, without limitation, those set forth in the National Center for Biotechnology Information (NCBI) databases at, for example, accession no Q8NCD3 (version Q8NCD3.2). For example, an increased level of a HJURP polypeptide (or an mRNA encoding a HJURP polypeptide) can be any level that is higher than a reference level of the HJURP polypeptide (or a reference level of an mRNA encoding a HJURP polypeptide). In some case, a reference level of a HJURP polypeptide in humans can be a level of a HJURP polypeptide in a sample (e.g., a tissue sample containing one or more melanoma cells) obtained from the human of from about 1 picogram (pg)/melanoma cell to about 2.3 pg/melanoma cell. For example, an increased level of a HJURP polypeptide can be at least 5% (e.g., about 5%, about 10%, about 20%, about 30%, about 40%, about 50%, about 60%, about 70%, about 80%, about 90%, about 95%, or more) higher than a reference level of a HJURP polypeptide. For example, an increased level of a HJURP polypeptide can be a level that is at least 2 (e.g., at least 5, at least 10, at least 15, at least 20, at least 25, at least 35, or at least 50) fold greater than a reference level of a HJURP polypeptide. In some case, a reference level of an mRNA encoding a HJURP polypeptide in humans can be a level of such mRNA that is from about 0.17 pg/melanoma cell to about 0.3 pg/melanoma cell. For example, an increased level of mRNA encoding a HJURP polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a HJURP polypeptide. For example, an increased level of mRNA encoding a HJURP polypeptide can be a level that is at least 2 (e.g., at least 5, at least 10, at least 15, at least 20, at least 25, at least 35, or at least 50) fold greater than a reference level of mRNA encoding a HJURP polypeptide.

In some cases, an altered level of a polypeptide can be an increased level of a NUF2 polypeptide. Examples of NUF2 polypeptides and nucleic acid sequences encoding a NUF2 polypeptide include, without limitation, those set forth in the NCBI databases at, for example, accession no. Q9BZD4 (version Q9BZD4.2). For example, an increased level of a NUF2 polypeptide (or an mRNA encoding a NUF2 polypeptide) can be any level that is higher than a reference level of the NUF2 polypeptide (or a reference level of an mRNA encoding a NUF2 polypeptide). In some case, a reference level of a NUF2 polypeptide in humans can be a level of a NUF2 polypeptide in a sample (e.g., a tissue sample containing one or more melanoma cells) obtained from the human of from about 0.9 pg/melanoma cell to about 1.5 pg/melanoma cell. For example, an increased level of a NUF2 polypeptide can be at least 5% (e.g., about 5%, about 10%, about 20%, about 30%, about 40%, about 50%, about 60%, about 70%, about 80%, about 90%, about 95%, or more) higher than a reference level of a NUF2 polypeptide. For example, an increased level of a NUF2 polypeptide can be a level that is at least 2 (e.g., at least 5, at least 10, at least 15, at least 20, at least 25, at least 35, or at least 50) fold greater than a reference level of a NUF2 polypeptide. In some case, a reference level of an mRNA encoding a NUF2 polypeptide in humans can be a level of such mRNA that is from about 0.11 pg/melanoma cell to about 0.19 pg/melanoma cell. For example, an increased level of mRNA encoding a NUF2 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a NUF2 polypeptide. For example, an increased level of mRNA encoding a NUF2 polypeptide can be a level that is at least 2 (e.g., at least 5, at least 10, at least 15, at least 20, at least 25, at least 35, or at least 50) fold greater than a reference level of mRNA encoding a NUF2 polypeptide.

In some cases, an altered level of a polypeptide can be an increased level of a PPPIR9A polypeptide. Examples of PPPIR9A polypeptides and nucleic acid sequences encoding a PPPIR9A polypeptide include, without limitation, those set forth in the NCBI databases at, for example, accession no. Q9ULJ8 (version Q9ULJ8.2). For example, an increased level of a PPPIR9A polypeptide (or an mRNA encoding a PPPIR9A polypeptide) can be any level that is higher than a reference level of the PPPIR9A polypeptide (or a reference level of an mRNA encoding a PPPIR9A polypeptide). In some case, a reference level of a PPPIR9A polypeptide in humans can be a level of a PPPIR9A polypeptide in a sample (e.g., a tissue sample containing one or more melanoma cells) obtained from the human of from about 1.3 pg/melanoma cell to about 2.6 pg/melanoma cell. For example, an increased level of a PPPIR9A polypeptide can be at least 5% (e.g., about 5%, about 10%, about 20%, about 30%, about 40%, about 50%, about 60%, about 70%, about 80%, about 90%, about 95%, or more) higher than a reference level of a PPPIR9A polypeptide. For example, an increased level of a PPPIR9A polypeptide can be a level that is at least 2 (e.g., at least 5, at least 10, at least 15, at least 20, at least 25, at least 35, or at least 50) fold greater than a reference level of a PPPIR9A polypeptide. In some case, a reference level of an mRNA encoding a PPPIR9A polypeptide in humans can be a level of such mRNA that is from about 0.37 pg/melanoma cell to about 0.77 pg/melanoma cell. For example, an increased level of mRNA encoding a PPPIR9A polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a PPPIR9A polypeptide. For example, an increased level of mRNA encoding a PPPIR9A polypeptide can be a level that is at least 2 (e.g., at least 5, at least 10, at least 15, at least 20, at least 25, at least 35, or at least 50) fold greater than a reference level of mRNA encoding a PPPIR9A polypeptide.

In some cases, an altered level of a polypeptide can be an increased level of a SRSF11 polypeptide. Examples of SRSF11 polypeptides and nucleic acid sequences encoding a SRSF11 polypeptide include, without limitation, those set forth in the NCBI databases at, for example, accession no. Q05519 (version Q05519.1). For example, an increased level of a SRSF11 polypeptide (or an mRNA encoding a SRSF11 polypeptide) can be any level that is higher than a reference level of the SRSF11 polypeptide (or a reference level of an mRNA encoding a SRSF11 polypeptide). In some case, a reference level of a SRSF11 polypeptide in humans can be a level of a SRSF11 polypeptide in a sample (e.g., a tissue sample containing one or more melanoma cells) obtained from the human of from about 3.2 pg/melanoma cell to about 7.0 pg/melanoma cell. For example, an increased level of a SRSF11 polypeptide can be at least 5% (e.g., about 5%, about 10%, about 20%, about 30%, about 40%, about 50%, about 60%, about 70%, about 80%, about 90%, about 95%, or more) higher than a reference level of a SRSF11 polypeptide. For example, an increased level of a SRSF11 polypeptide can be a level that is at least 2 (e.g., at least 5, at least 10, at least 15, at least 20, at least 25, at least 35, or at least 50) fold greater than a reference level of a SRSF11 polypeptide. In some case, a reference level of an mRNA encoding a SRSF11 polypeptide in humans can be a level of such mRNA that is from about 0.79 pg/melanoma cell to about 1.71 pg/melanoma cell. For example, an increased level of mRNA encoding a SRSF11 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a SRSF11 polypeptide. For example, an increased level of mRNA encoding a SRSF11 polypeptide can be a level that is at least 2 (e.g., at least 5, at least 10, at least 15, at least 20, at least 25, at least 35, or at least 50) fold greater than a reference level of mRNA encoding a SRSF11 polypeptide.

In some cases, an altered level of a polypeptide can be an increased level of a NT5DC3 polypeptide. Examples of NT5DC3 polypeptides and nucleic acid sequences encoding a NT5DC3 polypeptide include, without limitation, those set forth in the NCBI databases at, for example, accession no. Q86UY8 (version Q86UY8.1). For example, an increased level of a NT5DC3 polypeptide (or an mRNA encoding a NT5DC3 polypeptide) can be any level that is higher than a reference level of the NT5DC3 polypeptide (or a reference level of an mRNA encoding a NT5DC3 polypeptide). In some case, a reference level of a NT5DC3 polypeptide in humans can be a level of a NT5DC3 polypeptide in a sample (e.g., a tissue sample containing one or more melanoma cells) obtained from the human of from about 3.8 pg/melanoma cell to about 8.2 pg/melanoma cell. For example, an increased level of a NT5DC3 polypeptide can be at least 5% (e.g., about 5%, about 10%, about 20%, about 30%, about 40%, about 50%, about 60%, about 70%, about 80%, about 90%, about 95%, or more) higher than a reference level of a NT5DC3 polypeptide. For example, an increased level of a NT5DC3 polypeptide can be a level that is at least 2 (e.g., at least 5, at least 10, at least 15, at least 20, at least 25, at least 35, or at least 50) fold greater than a reference level of a NT5DC3 polypeptide. In some case, a reference level of an mRNA encoding a NT5DC3 polypeptide in humans can be a level of such mRNA that is from about 1.5 pg/melanoma cell to about 3.28 pg/melanoma cell. For example, an increased level of mRNA encoding a NT5DC3 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a NT5DC3 polypeptide. For example, an increased level of mRNA encoding a NT5DC3 polypeptide can be a level that is at least 2 (e.g., at least 5, at least 10, at least 15, at least 20, at least 25, at least 35, or at least 50) fold greater than a reference level of mRNA encoding a NT5DC3 polypeptide.

In some cases, an altered level of a polypeptide can be an increased level of a SERPINE2 polypeptide. Examples of SERPINE2 polypeptides and nucleic acid sequences encoding a SERPINE2 polypeptide include, without limitation, those set forth in the NCBI databases at, for example, accession no. P07093 (version P07093.1). For example, an increased level of a SERPINE2 polypeptide (or an mRNA encoding a SERPINE2 polypeptide) can be any level that is higher than a reference level of the SERPINE2 polypeptide (or a reference level of an mRNA encoding a SERPINE2 polypeptide). In some case, a reference level of a SERPINE2 polypeptide in humans can be a level of a SERPINE2 polypeptide in a sample (e.g., a tissue sample containing one or more melanoma cells) obtained from the human of from about 2.0 pg/melanoma cell to about 22.1 pg/melanoma cell. For example, an increased level of a SERPINE2 polypeptide can be at least 5% (e.g., about 5%, about 10%, about 20%, about 30%, about 40%, about 50%, about 60%, about 70%, about 80%, about 90%, about 95%, or more) higher than a reference level of a SERPINE2 polypeptide. For example, an increased level of a SERPINE2 polypeptide can be a level that is at least 2 (e.g., at least 5, at least 10, at least 15, at least 20, at least 25, at least 35, or at least 50) fold greater than a reference level of a SERPINE2 polypeptide. In some case, a reference level of an mRNA encoding a SERPINE2 polypeptide in humans can be a level of such mRNA that is from about 0.4 pg/melanoma cell to about 4.48 pg/melanoma cell. For example, an increased level of mRNA encoding a SERPINE2 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a SERPINE2 polypeptide. For example, an increased level of mRNA encoding a SERPINE2 polypeptide can be a level that is at least 2 (e.g., at least 5, at least 10, at least 15, at least 20, at least 25, at least 35, or at least 50) fold greater than a reference level of mRNA encoding a SERPINE2 polypeptide.

In some cases, an altered level of a polypeptide can be a decreased level of a PARM1 polypeptide. Examples of PARM1 polypeptides and nucleic acid sequences encoding a PARM1 polypeptide include, without limitation, those set forth in the NCBI databases at, for example, accession no. Q6UWI2 (version Q6UWI2.1). For example, a decreased level of a PARM1 polypeptide (or an mRNA encoding a PARM1 polypeptide) can be any level that is lower than a reference level of the PARM1 polypeptide (or a reference level of an mRNA encoding a PARM1 polypeptide). In some case, a reference level of a PARM1 polypeptide in humans can be a level of a PARM1 polypeptide in a sample (e.g., a tissue sample containing one or more melanoma cells) obtained from the human of from about 1.7 pg/melanoma cell to about 3.0 pg/melanoma cell. For example, a decreased level of a PARM1 polypeptide can be at least 5% (e.g., about 5%, about 10%, about 20%, about 30%, about 40%, about 50%, about 60%, about 70%, about 80%, about 90%, about 95%, or more) lower than a reference level of a PARM1 polypeptide. For example, a decreased level of a PARM1 polypeptide can be a level that is at least 2 (e.g., at least 5, at least 10, at least 15, at least 20, at least 25, at least 35, or at least 50) fold less than a reference level of a PARM1 polypeptide. In some case, a reference level of an mRNA encoding a PARM1 polypeptide in humans can be a level of such mRNA that is from about 0.93 pg/melanoma cell to about 1.65 pg/melanoma cell. For example, a decreased level of mRNA encoding a PARM1 polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a PARM1 polypeptide. For example, a decreased level of mRNA encoding a PARM1 polypeptide can be a level that is at least 2 (e.g., at least 5, at least 10, at least 15, at least 20, at least 25, at least 35, or at least 50) fold less than a reference level of mRNA encoding a PARM1 polypeptide.

In some cases, an altered level of a polypeptide can be a decreased level of an EMP2 polypeptide. Examples of EMP2 polypeptides and nucleic acid sequences encoding a EMP2 polypeptide include, without limitation, those set forth in the NCBI databases at, for example, accession no. P54851 (version P54851.1). For example, a decreased level of an EMP2 polypeptide (or an mRNA encoding an EMP2 polypeptide) can be any level that is lower than a reference level of the EMP2 polypeptide (or a reference level of an mRNA encoding an EMP2 polypeptide). In some case, a reference level of an EMP2 polypeptide in humans can be a level of an EMP2 polypeptide in a sample (e.g., a tissue sample containing one or more melanoma cells) obtained from the human of from about 0.6 pg/melanoma cell to about 1.5 pg/melanoma cell. For example, a decreased level of an EMP2 polypeptide can be at least 5% (e.g., about 5%, about 10%, about 20%, about 30%, about 40%, about 50%, about 60%, about 70%, about 80%, about 90%, about 95%, or more) lower than a reference level of an EMP2 polypeptide. For example, a decreased level of an EMP2 polypeptide can be a level that is at least 2 (e.g., at least 5, at least 10, at least 15, at least 20, at least 25, at least 35, or at least 50) fold less than a reference level of an EMP2 polypeptide. In some case, a reference level of an mRNA encoding an EMP2 polypeptide in humans can be a level of such mRNA that is from about 0.6 pg/melanoma cell to about 1.39 pg/melanoma cell. For example, a decreased level of mRNA encoding an EMP2 polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding an EMP2 polypeptide. For example, a decreased level of mRNA encoding an EMP2 polypeptide can be a level that is at least 2 (e.g., at least 5, at least 10, at least 15, at least 20, at least 25, at least 35, or at least 50) fold less than a reference level of mRNA encoding an EMP2 polypeptide.

In some cases, an altered level of a polypeptide can be a decreased level of a PRKCB polypeptide. Examples of PRKCB polypeptides and nucleic acid sequences encoding a PRKCB polypeptide include, without limitation, those set forth in the NCBI databases at, for example, accession no. P05771 (version P05771.4). For example, a decreased level of a PRKCB polypeptide (or an mRNA encoding a PRKCB polypeptide) can be any level that is lower than a reference level of the PRKCB polypeptide (or a reference level of an mRNA encoding a PRKCB polypeptide). In some case, a reference level of a PRKCB polypeptide in humans can be a level of a PRKCB polypeptide in a sample (e.g., a tissue sample containing one or more melanoma cells) obtained from the human of from about 2.9 pg/melanoma cell to about 5.2 pg/melanoma cell. For example, a decreased level of a PRKCB polypeptide can be at least 5% (e.g., about 5%, about 10%, about 20%, about 30%, about 40%, about 50%, about 60%, about 70%, about 80%, about 90%, about 95%, or more) lower than a reference level of a PRKCB polypeptide. For example, a decreased level of a PRKCB polypeptide can be a level that is at least 2 (e.g., at least 5, at least 10, at least 15, at least 20, at least 25, at least 35, or at least 50) fold less than a reference level of a PRKCB polypeptide. In some case, a reference level of an mRNA encoding a PRKCB polypeptide in humans can be a level of such mRNA that is about 1.05 pg/melanoma cell to about 1.89 pg/melanoma cell. For example, a decreased level of mRNA encoding a PRKCB polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a PRKCB polypeptide. For example, a decreased level of mRNA encoding a PRKCB polypeptide can be a level that is at least 2 (e.g., at least 5, at least 10, at least 15, at least 20, at least 25, at least 35, or at least 50) fold less than a reference level of mRNA encoding a PRKCB polypeptide.

In some cases, an altered level of a polypeptide can be a decreased level of a CD1E polypeptide. Examples of CD1E polypeptides and nucleic acid sequences encoding a CD1E polypeptide include, without limitation, those set forth in the NCBI databases at, for example, accession no. P15812 (version P15812.2). For example, a decreased level of a CD1E polypeptide (or an mRNA encoding a CD1E polypeptide) can be any level that is lower than a reference level of the CD1E polypeptide (or a reference level of an mRNA encoding a CD1E polypeptide). In some case, a reference level of a CD1E polypeptide in humans can be a level of a CD1E polypeptide in a sample (e.g., a tissue sample containing one or more melanoma cells) obtained from the human of from about 5.9 pg/melanoma cell to about 7.7 pg/melanoma cell. For example, a decreased level of a CD1E polypeptide can be at least 5% (e.g., about 5%, about 10%, about 20%, about 30%, about 40%, about 50%, about 60%, about 70%, about 80%, about 90%, about 95%, or more) lower than a reference level of a CD1E polypeptide. For example, a decreased level of a CD1E polypeptide can be a level that is at least 2 (e.g., at least 5, at least 10, at least 15, at least 20, at least 25, at least 35, or at least 50) fold less than a reference level of a CD1E polypeptide. In some case, a reference level of an mRNA encoding a CD1E polypeptide in humans can be a level of such mRNA that is from about 0.89 pg/melanoma cell to about 1.17 pg/melanoma cell. For example, a decreased level of mRNA encoding a CD1E polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a CD1E polypeptide. For example, a decreased level of mRNA encoding a CD1E polypeptide can be a level that is at least 2 (e.g., at least 5, at least 10, at least 15, at least 20, at least 25, at least 35, or at least 50) fold less than a reference level of mRNA encoding a CD1E polypeptide.

In some cases, an altered level of a polypeptide can be a decreased level of an ALDH2 polypeptide. Examples of ALDH2 polypeptides and nucleic acid sequences encoding an ALDH2 polypeptide include, without limitation, those set forth in the NCBI databases at, for example, accession no. P05091 (version P05091.2). For example, a decreased level of an ALDH2 polypeptide (or an mRNA encoding an ALDH2 polypeptide) can be any level that is lower than a reference level of the ALDH2 polypeptide (or a reference level of an mRNA encoding an ALDH2 polypeptide). In some case, a reference level of an ALDH2 polypeptide in humans can be a level of an ALDH2 polypeptide in a sample (e.g., a tissue sample containing one or more melanoma cells) obtained from the human of from about 4.8 pg/melanoma cell to about 11.4 pg/melanoma cell. For example, a decreased level of an ALDH2 polypeptide can be at least 5% (e.g., about 5%, about 10%, about 20%, about 30%, about 40%, about 50%, about 60%, about 70%, about 80%, about 90%, about 95%, or more) lower than a reference level of an ALDH2 polypeptide. For example, a decreased level of an ALDH2 polypeptide can be a level that is at least 2 (e.g., at least 5, at least 10, at least 15, at least 20, at least 25, at least 35, or at least 50) fold less than a reference level of an ALDH2 polypeptide. In some case, a reference level of an mRNA encoding an ALDH2 polypeptide in humans can be a level of such mRNA that is from about 2.87 pg/melanoma cell to about 6.76 pg/melanoma cell. For example, a decreased level of mRNA encoding an ALDH2 polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding an ALDH2 polypeptide. For example, a decreased level of mRNA encoding an ALDH2 polypeptide can be a level that is at least 2 (e.g., at least 5, at least 10, at least 15, at least 20, at least 25, at least 35, or at least 50) fold less than a reference level of ALDH2 encoding an EMP2 polypeptide.

In some cases, an altered level of a polypeptide can be a decreased level of a CD200R1 polypeptide. Examples of CD200R1 polypeptides and nucleic acid sequences encoding a CD200R1 polypeptide include, without limitation, those set forth in the NCBI databases at, for example, accession no. Q8TD46 (version Q8TD46.3). For example, a decreased level of a CD200R1 polypeptide (or an mRNA encoding a CD200R1 polypeptide) can be any level that is lower than a reference level of the CD200R1 polypeptide (or a reference level of an mRNA encoding a CD200R1 polypeptide). In some case, a reference level of a CD200R1 polypeptide in humans can be a level of a CD200R1 polypeptide in a sample (e.g., a tissue sample containing one or more melanoma cells) obtained from the human of from about 1.1 pg/melanoma cell to about 1.8 pg/melanoma cell. For example, a decreased level of a CD200R1 polypeptide can be at least 5% (e.g., about 5%, about 10%, about 20%, about 30%, about 40%, about 50%, about 60%, about 70%, about 80%, about 90%, about 95%, or more) lower than a reference level of a CD200R1 polypeptide. For example, a decreased level of a CD200R1 polypeptide can be a level that is at least 2 (e.g., at least 5, at least 10, at least 15, at least 20, at least 25, at least 35, or at least 50) fold less than a reference level of a CD200R1 polypeptide. In some case, a reference level of an mRNA encoding a CD200R1 polypeptide in humans can be a level of such mRNA that is from about 0.36 pg/melanoma cell to about 0.59 pg/melanoma cell. For example, a decreased level of mRNA encoding a CD200R1 polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a CD200R1 polypeptide. For example, a decreased level of mRNA encoding a CD200R1 polypeptide can be a level that is at least 2 (e.g., at least 5, at least 10, at least 15, at least 20, at least 25, at least 35, or at least 50) fold less than a reference level of mRNA encoding a CD200R1 polypeptide.

Any appropriate method can be used to identify the presence, absence, or level of a polypeptide or mRNA encoding that polypeptide. In some cases, the presence, absence, or level of an mRNA encoding a polypeptide can be assessed by detecting and/or quantifying the mRNA encoding a polypeptide. Examples of methods that can be used to detect and/or quantify mRNA include, without limitation, RT-PCR techniques (e.g., quantitative RT-PCR techniques), and RNA sequencing methods. In some cases, the presence, absence, or level of a polypeptide can be assessed by detecting and/or quantifying the polypeptide. Examples of methods that can be used to detect and/or quantify polypeptides include, without limitation, immunohistochemistry (IHC) techniques, mass spectrometry techniques (e.g., proteomics-based mass spectrometry assays or targeted quantification-based mass spectrometry assays), western blotting techniques, and enzyme-linked immunoassay (ELISA). In some cases, the presence, absence, or level of a polypeptide and/or mRNA encoding that polypeptide can be identified as described in Example 1.

Any appropriate sample from a mammal (e.g., a human) having melanoma (e.g., PCM) can be assessed as described herein (e.g., for the presence or absence of an altered level of 4 or more polypeptides). In some cases, a sample can be a biological sample. In some cases, a sample can contain one or more cancer cells (e.g., melanoma cells). In some cases, a sample can contain one or more biological molecules (e.g., nucleic acids such as DNA and RNA, polypeptides, carbohydrates, lipids, hormones, and/or metabolites). For example, tissue samples (e.g., tissue samples obtained by skin biopsy). A sample can be a fresh sample or a fixed sample (e.g., a formaldehyde-fixed sample or a formalin-fixed sample). In some cases, one or more biological molecules can be isolated from a sample (e.g., from one or more cancer cells within the sample). For example, nucleic acid can be isolated from a sample and can be assessed as described herein. For example, polypeptides can be isolated from a sample and can be assessed as described herein.

In some cases, methods and materials for determining whether or not a melanoma (e.g., a PCM) is likely to relapse (e.g., likely to relapse following treatment) as described herein also can include determining a Breslow depth of the melanoma. A Breslow depth of a melanoma can be from about 0.1 mm to about 10 mm. In some cases, a Breslow depth of greater than about 0.8 mm (e.g., from about 0.8 mm to about 10 mm, from about 0.8 mm to about 9 mm, from about 0.8 mm to about 8 mm, from about 0.8 mm to about 7 mm, from about 0.8 mm to about 6 mm, from about 0.8 mm to about 5 mm, from about 0.8 mm to about 4 mm, from about 0.8 mm to about 3 mm, from about 0.8 mm to about 2 mm, from about 0.8 mm to about 1 mm, from about 1 mm to about 10 mm, from about 2 mm to about 10 mm, from about 3 mm to about 10 mm, from about 4 mm to about 10 mm, from about 5 mm to about 10 mm, from about 6 mm to about 10 mm, from about 7 mm to about 10 mm, from about 8 mm to about 10 mm, from about 9 mm to about 10 mm, from about 1 mm to about 9 mm, from about 2 mm to about 8 mm, from about 3 mm to about 7 mm, from about 4 mm to about 6 mm, from about 1 mm to about 3 mm, from about 2 mm to about 4 mm, from about 3 mm to about 5 mm, from about 4 mm to about 6 mm, from about 5 mm to about 7 mm, from about 6 mm to about 8 mm, or from about 7 mm to about 9 mm) can indicate that a melanoma is likely to relapse. For example, a Breslow depth of greater than about 0.8 mm in combination with the presence of an altered level (e.g., an increased level or a decreased level) of 4 or more polypeptides can indicate that a melanoma is likely to relapse. In some cases, a Breslow depth of less than about 0.8 mm (e.g., from about 0.1 mm to about 0.7 mm, from about 0.1 mm to about 0.6 mm, from about 0.1 mm to about 0.5 mm, from about 0.1 mm to about 0.4 mm, from about 0.1 mm to about 0.3 mm, from about 0.1 mm to about 0.2 mm, from about 0.2 mm to about 0.8 mm, from about 0.3 mm to about 0.8 mm, from about 0.4 mm to about 0.8 mm, from about 0.5 mm to about 0.8 mm, from about 0.6 mm to about 0.8 mm, from about 0.7 mm to about 0.8 mm, from about 0.2 mm to about 0.7 mm, from about 0.3 mm to about 0.6 mm, from about 0.4 mm to about 0.5 mm, from about 0.2 mm to about 0.4 mm, from about 0.3 mm to about 0.5 mm, from about 0.4 mm to about 0.6 mm, or from about 0.5 mm to about 0.7 mm) can indicate that a melanoma is not likely to relapse. For example, a Breslow depth of less than about 0.8 mm in combination with the absence of an altered level (e.g., an increased level or a decreased level) of 4 or more polypeptides can indicate that a melanoma is not likely to relapse. Any appropriate method can be used to determine a Breslow depth of a melanoma (e.g., a PCM). For example, tumor depth of a skin sample (e.g., a skin sample obtained by excisional biopsy), an ocular micrometer at a right angle to the skin, and/or measurements obtained from digitized slides using computer software can be used to determine a Breslow depth of a melanoma (e.g., a PCM).

In some cases, a mammal (e.g., a human) having a melanoma (e.g., a PCM) that is identified as not being likely to relapse (e.g., not likely to relapse following treatment) as described herein (e.g., based, at least in part, on the absence of an altered level of 4 or more polypeptides) can be selected to receive surgery to remove the melanoma. For example, a mammal having a melanoma (e.g., a PCM) that is identified as lacking an increased level of a HJURP polypeptide (or mRNA encoding a HJURP polypeptide), an increased level of a NUF2 polypeptide (or mRNA encoding a NUF2 polypeptide), an increased level of a PPPIR9A polypeptide (or mRNA encoding a PPPIR9A polypeptide), an increased level of a SRSF11 polypeptide (or mRNA encoding a SRSF11 polypeptide), an increased level of a NT5DC3 polypeptide (or mRNA encoding a NT5DC3 polypeptide), an increased level of SERPINE2 polypeptide (or mRNA encoding a SERPINE2 polypeptide), a decreased level of a PARM1 polypeptide (or mRNA encoding a PARM1 polypeptide), a decreased level of an EMP2 polypeptide (or mRNA encoding an EMP2 polypeptide), a decreased level of a PRKCB polypeptide (or mRNA encoding a PRKCB polypeptide), a decreased level of a CD1E polypeptide (or mRNA encoding a CD1E polypeptide), a decreased level of an ALDH2 polypeptide (or mRNA encoding an ALDH2 polypeptide), and a decreased level of CD200R1 polypeptide (or mRNA encoding a CD200R1 polypeptide) in a sample (e.g., a tissue sample containing one or more melanoma cells) obtained from the mammal can be selected to undergo surgery to remove the melanoma (e.g., in the absence of any adjuvant therapies).

In some cases, a mammal (e.g., a human) having a melanoma (e.g., a PCM) that is identified as being likely to relapse (e.g., likely to relapse following treatment) as described herein (e.g., based, at least in part, on the presence of an altered level of 4 or more polypeptides) can be selected to receive surgery to remove the melanoma and can be selected for increased monitoring. For example, a mammal having a melanoma (e.g., a PCM) that is identified as having the presence of an altered level of 4 or more polypeptides in a sample (e.g., a tissue sample containing one or more melanoma cells) obtained from the mammal can be selected to receive one or more (e.g., one, two, three, four, five, or more) tests that can be used to identify whether or not a melanoma (e.g., PCM) has relapsed.

In some cases, a mammal (e.g., a human) having a melanoma (e.g., a PCM) that is identified as being likely to relapse (e.g., likely to relapse following treatment) as described herein (e.g., based, at least in part, on the presence of an altered level of 4 or more polypeptides) can be selected to receive surgery to remove the melanoma and can be selected for treatment with one or more adjuvant therapies. For example, a mammal having a melanoma (e.g., a PCM) that is identified as having the presence of an altered level of 4 or more polypeptides in a sample (e.g., a tissue sample containing one or more melanoma cells) obtained from the mammal can be selected to undergo surgery to remove the melanoma and to receive one or more (e.g., one, two, three, four, five, or more) adjuvant therapies.

This document also provides methods for treating a mammal (e.g., a human) having melanoma (e.g., a PCM). In some cases, a mammal (e.g., a human) having a melanoma (e.g., a PCM) that is assessed as described herein (e.g., to determine whether or not the melanoma is likely to relapse based, at least in part, on the presence or absence of an altered level of 4 or more polypeptides in a sample obtained from the mammal) can be administered or instructed to self-administer one or more (e.g., one, two, three, four, five, or more) cancer treatments, where the one or more cancer treatments are effective to treat the cancer within the mammal. For example, a mammal having melanoma (e.g., a PCM) can be administered or instructed to self-administer one or more cancer treatments selected based, at least in part, on whether or not the melanoma is likely to relapse (e.g., based, at least in part, on the presence or absence of an altered level of 4 or more polypeptides in a sample obtained from the mammal).

In general, a cancer treatment for melanoma (e.g., a PCM) can include any appropriate melanoma cancer treatment. In some cases, a cancer treatment for melanoma (e.g., a PCM) can include surgery and other medical interventions. Examples of surgeries and other medical interventions that can be performed on a mammal having melanoma (e.g., a PCM) to treat the mammal include, without limitation, surgery (e.g., to remove a melanoma, to remove the tissue around a melanoma, to remove a melanoma and the surrounding tissue, and/or to remove regional lymph nodes), and radiation therapy. In some cases, a cancer treatment can include administering one or more anti-cancer drugs (e.g., chemotherapeutic agents, targeted cancer drugs, and immunotherapy drugs) to a mammal in need thereof. Examples of anti-cancer drugs that can be administered to a mammal having melanoma (e.g., a PCM) can include, without limitation, nivolumab (e.g., OPDIVO®), ipilimumab (e.g., YERVOY®), pembrolizumab (e.g., KEYTRUDA®), talimogene laherparepvec (T-VEC; e.g., IMLYGIC®), vemurafenib (e.g., ZELBORAF®), dabrafenib (e.g., TAFINLAR®), encorafenib (e.g., BRAFTOVI®), trametinib (e.g., MEKINIST®), cobimetinib (e.g., COTELLIC®), binimetinib (e.g., MEKTOVI®), atezolizumab (e.g., TECENTRIQ®), avelumab (e.g., BAVENCIO®), durvalumab (e.g., IMFINZI®), and combinations thereof.

When treating a mammal (e.g., a human) having a melanoma (e.g., a PCM) that is identified as not being likely to relapse (e.g., not likely to relapse following treatment) as described herein (e.g., based, at least in part, on the absence of an altered level of 4 or more polypeptides), the mammal can undergo surgery (e.g., local re-excision surgery) to remove the melanoma. For example, a mammal having a melanoma (e.g., a PCM) that is identified as lacking an increased level of a HJURP polypeptide (or mRNA encoding a HJURP polypeptide), an increased level of a NUF2 polypeptide (or mRNA encoding a NUF2 polypeptide), an increased level of a PPP1R9A polypeptide (or mRNA encoding a PPPIR9A polypeptide), an increased level of a SRSF11 polypeptide (or mRNA encoding a SRSF11 polypeptide), an increased level of a NT5DC3 polypeptide (or mRNA encoding a NT5DC3 polypeptide), an increased level of SERPINE2 polypeptide (or mRNA encoding a SERPINE2 polypeptide), a decreased level of a PARM1 polypeptide (or mRNA encoding a PARM1 polypeptide), a decreased level of an EMP2 polypeptide (or mRNA encoding an EMP2 polypeptide), a decreased level of a PRKCB polypeptide (or mRNA encoding a PRKCB polypeptide), a decreased level of a CD1E polypeptide (or mRNA encoding a CD1E polypeptide), a decreased level of an ALDH2 polypeptide (or mRNA encoding an ALDH2 polypeptide), and a decreased level of CD200R1 polypeptide (or mRNA encoding a CD200R1 polypeptide) in a sample (e.g., a tissue sample containing one or more melanoma cells) obtained from the mammal can undergo local re-excision surgery to remove the melanoma.

In some cases, when treating a mammal (e.g., a human) having a melanoma (e.g., a PCM) that is identified as not being likely to relapse (e.g., not likely to relapse following treatment) as described herein (e.g., based, at least in part, on the absence of an altered level of 4 or more polypeptides), the surgery can be the sole cancer treatment used to treat the mammal. For example, a mammal having a melanoma (e.g., a PCM) that is identified as lacking an increased level of a HJURP polypeptide (or mRNA encoding a HJURP polypeptide), an increased level of a NUF2 polypeptide (or mRNA encoding a NUF2 polypeptide), an increased level of a PPPIR9A polypeptide (or mRNA encoding a PPPIR9A polypeptide), an increased level of a SRSF11 polypeptide (or mRNA encoding a SRSF11 polypeptide), an increased level of a NT5DC3 polypeptide (or mRNA encoding a NT5DC3 polypeptide), an increased level of SERPINE2 polypeptide (or mRNA encoding a SERPINE2 polypeptide), a decreased level of a PARM1 polypeptide (or mRNA encoding a PARM1 polypeptide), a decreased level of an EMP2 polypeptide (or mRNA encoding an EMP2 polypeptide), a decreased level of a PRKCB polypeptide (or mRNA encoding a PRKCB polypeptide), a decreased level of a CD1E polypeptide (or mRNA encoding a CD1E polypeptide), a decreased level of an ALDH2 polypeptide (or mRNA encoding an ALDH2 polypeptide), and a decreased level of CD200R1 polypeptide (or mRNA encoding a CD200R1 polypeptide) in a sample (e.g., a tissue sample containing one or more melanoma cells) obtained from the mammal can undergo surgery to remove the melanoma and is not administered or instructed to self-administer any additional (e.g., adjuvant) cancer treatments.

When treating a mammal (e.g., a human) having a melanoma (e.g., a PCM) that is identified as being likely to relapse (e.g., likely to relapse following treatment) as described herein (e.g., based, at least in part, on the presence of an altered level of 4 or more polypeptides), the mammal can undergo surgery to remove the melanoma and can be administered or instructed to self-administer one or more adjuvant therapies. For example, a mammal having a melanoma (e.g., a PCM) that is identified as having the presence of an altered level of 4 or more polypeptides in a sample (e.g., a tissue sample containing one or more melanoma cells) obtained from the mammal can undergo surgery to remove the melanoma and can be administered or instructed to self-administer one or more (e.g., one, two, three, four, five, or more) adjuvant therapies. An adjuvant therapy can be any appropriate cancer treatment. In some cases, radiation therapy can be used as an adjuvant therapy with surgery to treat a mammal having a melanoma (e.g., a PCM) that is identified as being likely to relapse as described herein. In some cases, a cancer treatment that can be used as an adjuvant therapy with surgery to treat a mammal having a melanoma (e.g., a PCM) that is identified as being likely to relapse as described herein can include administering one or more anti-cancer drugs (e.g., chemotherapeutic agents, targeted cancer drugs, and immunotherapy drugs) to the mammal. Examples of anti-cancer drugs that can be administered to a mammal having melanoma (e.g., a PCM) can include, without limitation, nivolumab (e.g., OPDIVO®), ipilimumab (e.g., YERVOY®), pembrolizumab (e.g., KEYTRUDA®), talimogene laherparepvec (T-VEC; e.g., IMLYGIC®), vemurafenib (e.g., ZELBORAF®), dabrafenib (e.g., TAFINLAR®), encorafenib (e.g., BRAFTOVI®), trametinib (e.g., MEKINIST®), cobimetinib (e.g., COTELLIC®), binimetinib (e.g., MEKTOVI®), atezolizumab (e.g., TECENTRIQ®), avelumab (e.g., BAVENCIO®), durvalumab (e.g., IMFINZI®), and combinations thereof.

In some cases, when treating a mammal (e.g., a human) having melanoma (e.g., PCM) as described herein, the treatment can be effective to treat the melanoma. For example, the number of cancer cells present within a mammal can be reduced using the methods and materials described herein. In some cases, the methods and materials described herein can be used to reduce the number of cancer cells present within a mammal having melanoma by, for example, 10, 20, 30, 40, 50, 60, 70, 80, 90, 95, or more percent. In some cases, the number of cancer cells present within a mammal does not increase. For example, the size (e.g., volume) of one or more tumors present within a mammal can be reduced using the methods and materials described herein. In some cases, the methods and materials described herein can be used to reduce the size of one or more tumors present within a mammal having melanoma (e.g., PCM) by, for example, 10, 20, 30, 40, 50, 60, 70, 80, 90, 95, or more percent. In some cases, the size (e.g., volume) of one or more tumors present within a mammal does not increase.

In some cases, when treating a mammal (e.g., a human) having melanoma (e.g., PCM) as described herein, the treatment can be effective to treat the melanoma while minimizing the risk of relapse. For example, the number of cancer cells present within a mammal can be reduced using the methods and materials described herein. In some cases, the methods and materials described herein can be used to reduce the number of cancer cells present within a mammal having melanoma by, for example, 10, 20, 30, 40, 50, 60, 70, 80, 90, 95, or more percent. In some cases, the number of cancer cells present within a mammal does not increase. For example, the size (e.g., volume) of one or more tumors present within a mammal can be reduced using the methods and materials described herein. In some cases, the methods and materials described herein can be used to reduce the size of one or more tumors present within a mammal having melanoma (e.g., PCM) by, for example, 10, 20, 30, 40, 50, 60, 70, 80, 90, 95, or more percent. In some cases, the size (e.g., volume) of one or more tumors present within a mammal does not increase.

In some cases, when treating a mammal (e.g., a human) having melanoma (e.g., PCM) as described herein, the treatment can be effective to improve survival of the mammal. For example, the methods and materials described herein can be used to improve disease-free survival (e.g., relapse-free survival). For example, the methods and materials described herein can be used to improve overall survival. For example, the methods and materials described herein can be used to improve the survival of a mammal having melanoma (e.g., PCM) by, for example, 10, 20, 30, 40, 50, 60, 70, 80, 90, 95, or more percent. For example, the methods and materials described herein can be used to improve the survival of a mammal having melanoma (e.g., PCM) by, for example, at least 6 months (e.g., about 6 months, about 8 months, about 10 months, about 1 year, about 1.5 years, about 2 years, about 2.5 years, or about 3 years).

In some cases, when treating a mammal (e.g., a human) having melanoma (e.g., PCM) as described herein, the treatment can be effective to improve survival of the mammal while minimizing the risk of relapse. For example, the methods and materials described herein can be used to improve disease-free survival (e.g., relapse-free survival). For example, the methods and materials described herein can be used to improve overall survival. For example, the methods and materials described herein can be used to improve the survival of a mammal having melanoma (e.g., PCM) by, for example, 10, 20, 30, 40, 50, 60, 70, 80, 90, 95, or more percent. For example, the methods and materials described herein can be used to improve the survival of a mammal having melanoma (e.g., PCM) by, for example, at least 6 months (e.g., about 6 months, about 8 months, about 10 months, about 1 year, about 1.5 years, about 2 years, about 2.5 years, or about 3 years).

In some cases, when treating a mammal (e.g., a human) having melanoma (e.g., PCM) as described herein, the treatment can be effective to reduce or eliminate one or more symptoms of the melanoma. Examples of symptoms of melanoma (e.g., PCM) that can be reduced or eliminated using the methods and materials described herein can include, without limitation, changes in one or more existing moles, development of a new pigmented and/or unusual-looking growth on the skin, sores that don't heal, redness and/or swelling that spreads outside the border of a spot to the surrounding skin, itchiness, tenderness, pain, blurry vision, partial loss of sight, and dark spots in the iris. For example, the methods and materials described herein can be used to reduce one or more symptoms of melanoma (e.g., PCM) within a mammal having melanoma by, for example, 10, 20, 30, 40, 50, 60, 70, 80, 90, 95, or more percent.

In some cases, when treating a mammal (e.g., a human) having melanoma (e.g., PCM) as described herein, the treatment can be effective to reduce or eliminate one or more symptoms of the melanoma while minimizing the risk of relapse. Examples of symptoms of melanoma (e.g., PCM) that can be reduced or eliminated using the methods and materials described herein can include, without limitation, changes in one or more existing moles, development of a new pigmented and/or unusual-looking growth on the skin, sores that don't heal, redness and/or swelling that spreads outside the border of a spot to the surrounding skin, itchiness, tenderness, pain, blurry vision, partial loss of sight, and dark spots in the iris. For example, the methods and materials described herein can be used to reduce one or more symptoms of melanoma (e.g., PCM) within a mammal having melanoma by, for example, 10, 20, 30, 40, 50, 60, 70, 80, 90, 95, or more percent.

In some cases, a course of treatment, the number of cancer cells present within a mammal and/or the severity of one or more symptoms related to the melanoma (e.g., PCM) can be monitored. Any appropriate method can be used to determine whether or not the number of cancer cells present within a mammal is reduced. For example, imaging techniques can be used to assess the number of cancer cells present within a mammal.

The invention will be further described in the following examples, which do not limit the scope of the invention described in the claims.

### EXAMPLES

### Example 1: A Prognostic Gene Expression Signature for Primary Cutaneous Melanoma

While the melanoma biomarker landscape is evolving, there are currently no generally accepted, thoroughly studied, guideline-recommended predictive or prognostic biomarkers available that identify early-stage high-risk disease (Grossman et al., Melanoma Management, 6: MMT32 (2019); and Grossman et al., JAMA Dermatol., 156:1004-11 (2020)).

This Example describes the identification of a gene expression signature for primary cutaneous melanoma that can be used to predict whether or not a melanoma patient is at a higher risk of relapse.

### Discovery Cohort

For the discovery of the classifier, a subset of an 837 patient, prospectively-designed archival discovery cohort was used. This cohort has been characterized as described elsewhere (Eggermont et al., Eur. J. Cancer, 140:11-8 (2020); Bellomo et al., JCO Precision Oncology, 2020:319-34 (2020); Meves et al., J. Clin. Oncol., 33:2509-15 (2015); Kung et al., J. Invest. Dermatol., 140:2301-4.e3 (2020); Quattrocchi et al., Int. J. Dermatol., 59:1241-8 (2020); and Meves et al., Mayo Clin. Proc. Innov. Qual. Outcomes, 4:586-7 (2020)). Briefly, this is a cohort of melanoma patients who had a SLNb performed within 90 days of their diagnosis (a time interval shown not to affect SLNb positivity or survival rates). Electronic searches of pathology reports retrospectively identified patients with primary cutaneous melanoma with known SLNb status.

### Eligibility

Eligibility was based on histopathology data derived from patient medical records and established by two or more board-certified dermatopathologists. Inclusion was determined by institutional practice guidelines for recommending SLNb, which were based on Breslow thickness, ulceration, mitoses, and patient age. Patients were eligible for this study if they met one of the following three conditions: 1) Breslow thickness greater than 1.0 mm; 2) Breslow thickness of 0.75-0.99 mm and presence of ulceration, mitoses, or patient age less than 40 years; or 3) Breslow thickness of 0.50-0.74 mm and presence of at least two of the following: ulceration, mitoses and patient age less than 40 years.

### Exclusion Criteria

Exclusion criteria were as follows: M1 disease within 90 days of primary diagnosis; insufficient primary tumor diagnostic biopsy tissue; inadequate RNA harvested and, denial of access to medical records for research purposes. Patients were not excluded based on histologic type, but histologic type was recorded as a clinicopathologic variable.

### Patient and Tumor Characteristics

Patient and tumor characteristics are as described elsewhere (Eggermont et al., Eur. J. Cancer, 140:11-8 (2020)). Most patients, namely 580/837 or 69%, were diagnosed with early-stage I/IIA disease. 50/837 patients or 6% developed brain metastases. 78/837 or 9% of patients presented with stage IIIA disease, 42 of these 78 patients had low-volume SLNb metastasis (individual tumor cells or cell clusters <0.1 mm).

### RNA Sequencing

RNA sequencing data, which was used for the discovery of the disclosed CP-GEP classifier, was derived from a subset of 382 of the 837 patients:
384 samples were selected from the 837-patient cohort based on RNA quality, i.e., RIN scores, DV200 scores, concentration, fragment sizes, and the presence of primer dimers.
Relapse-free survival (RFS): The majority of patients (52%) in the RNAseq group were diagnosed with stage I/IIA disease (199/384). Median follow-up was 43.7 months; 87/384 (23%) of patients experienced a relapse as of Dec. 1, 2019.
RNA libraries were prepared according to the manufacturer's instructions for the TruSeq RNA Exome Library Prep Kit by Illumina (San Diego, CA).
Coding regions of the transcriptome were captured by pooling four of the cDNA libraries at 200 ng each, following the TruSeq® RNA Exome Library Prep Kit instructions.
Libraries were multiplexed at 96 samples per flow cell, following Illumina's standard protocol for the Illumina NovaSeq™ 6000 and S4 flow cell. The flow cells were sequenced as 100×2 paired-end reads using the NovaSeq™ S4 sequencing kit and NovaSeq™ Control Software v1.6.0. Base-calling was performed using Illumina's RTA version 3.4.4.
An S4 flow cell's total output is nine billion single-end reads, or 18 billion paired-end reads passing filters (about 94 million single-end reads per sample).

### RNA Sequencing Data—Quality Control

Quality Control (QC) was performed using MultiQC.
Total samples sequenced: 384.
Sample MM725 failed sequencing.
The remaining 383 samples showed the following characteristics:
   Average sequencing depth (million reads): 91 [range: 13.5-502].
   Average mapped reads (%): 81 [range: 71-87]; this means that on average, ˜19% of reads were unmapped, which falls within the normal, expected range.
   Average ribosomal reads (%): 0.57 [range: 0.1-1.8]; this is excellent; no reads were lost to ribosomal regions.
Included into downstream analysis: 383 of 384 total samples.
Overall, the QC of the samples looked excellent.
   Univariate Association of Gene Expression with Relapse-Free Survival.

The primary endpoint here was time to relapse after initial diagnosis of melanoma. Subjects who died, were lost to follow-up, or reached the end of the study period without relapse were censored at those respective time points.

Initial analyses examined, in turn, the univariate associations of clinical and gene expression attributes with time to relapse using Cox proportional hazards regression analysis. Clinical characteristics included age at diagnosis (years), gender, Breslow depth (mm), presence of tumor ulceration and mitotic rate level. Gene expression was examined first using the original CPM expression measures and second using inverse normal-transformed (van der Waerden) rank measures. All continuous variables were modeled assuming a log-linear association with time to relapse.
Relapse events: 95/383; median follow-up: 4.71 years.
A 9,950 out of a total of 17,450 sequenced unique genes (57%) were univariately associated with melanoma relapse as defined by a q-value<0.05.
The q-value is an adjusted p-value, taking in to account the false discovery rate (FDR).
   Multivariate Analysis: Association of Gene Expression with Relapse-Free Survival
Multivariate=each expression association is adjusted for the independent effects of patient age at diagnosis, sex, ulcerations, Breslow depth and mitotic rate level.
For the categorical variables, males were compared to females (referent group) and to those with an ulceration to those without (referent group). All the continuous variables are modeled as a one degree-of-freedom linear term, which assumes a log-linear association between the variable of interest and risk of relapse. By transforming the expression variables using inverse normal (van der Waerden) scores, hazard ratios for these variables can now be interpreted as the change in risk of relapse per one standard deviation, or z-score, in the transformed value of the expression variable.
3,514 of 17,450 genes (20%) are significant based on multivariate analysis using a q-value cutoff of <5%.
3,345 of 17,450 genes (19%) are significant in both univariate and multivariate analysis, based on the q-value cutoff (5% of significant tests will result in false positives). 19% of the genes passed the cutoff.

### Genes Expression Biomarkers Currently Used for the Merlin Assay by SkylineDx

Eight primary genes, which are used as biomarkers in the Merlin assay by SkylineDx can be found in Table 1.

TABLE 1

| | | hazard | | | | qVal | hazard |
|---|---|---|---|---|---|---|---|
| Var | GeneName | ratio_Uni | lower_CI_95_Uni | upper_CI_95_Uni | pValue_Uni | Univariate | ratio_Multi |
| ENSG00000106799 | TGFBR1 | 1.592103 | 1.292089 | 1.961777 | 1.27E−05 | 3.26E−05 | 1.468639 |
| ENSG00000118785 | SPP1 | 1.945038 | 1.565601 | 2.416436 | 1.87E−09 | 3.94E−08 | 1.530689 |
| ENSG00000135919 | SERPINE2 | 1.780361 | 1.447634 | 2.189563 | 4.64E−08 | 3.81E−07 | 1.445369 |
| ENSG00000169429 | CXCL8 | 2.034302 | 1.624003 | 2.548263 | 6.45E−10 | 1.87E−08 | 1.523969 |
| ENSG00000130513 | GDF15 | 1.530307 | 1.242484 | 1.884806 | 6.27E−05 | 0.000122 | 1.318751 |
| ENSG00000104368 | PLAT | 1.625644 | 1.310651 | 2.016341 | 9.79E−06 | 2.63E−05 | 1.327797 |
| ENSG00000120215 | MLANA | 1.352819 | 1.096297 | 1.669364 | 0.004847 | 0.004586 | 1.25802 |
| ENSG00000259753 | ITGB3 | 1.684214 | 1.354164 | 2.094707 | 2.81E−06 | 9.71E−06 | 1.275639 |
| ENSG00000259207 | ITGB3 | 1.664098 | 1.339418 | 2.067482 | 4.25E−06 | 1.35E−05 | 1.260363 |
| ENSG00000138131 | LOXL4 | 1.306431 | 1.052233 | 1.622039 | 0.01547 | 0.01208 | 1.195978 |

| | | | | | qVal |
|---|---|---|---|---|---|
| Var | GeneName | lower_CI_95_Multi | upper_CI_95_Multi | pValue_Multi | Multivariate |
| ENSG00000106799 | TGFBR1 | 1.166196 | 1.849519 | 0.001088 | 0.010027 |
| ENSG00000118785 | SPP1 | 1.169293 | 2.003784 | 0.001947 | 0.013622 |
| ENSG00000135919 | SERPINE2 | 1.139969 | 1.832586 | 0.002352 | 0.014988 |
| ENSG00000169429 | CXCL8 | 1.116991 | 2.079232 | 0.007861 | 0.028221 |
| ENSG00000130513 | GDF15 | 1.065749 | 1.631815 | 0.010899 | 0.034475 |
| ENSG00000104368 | PLAT | 1.053158 | 1.674055 | 0.016481 | 0.043946 |
| ENSG00000120215 | MLANA | 1.016803 | 1.556463 | 0.034565 | 0.068016 |
| ENSG00000259753 | ITGB3 | 1.010145 | 1.610913 | 0.040877 | 0.075533 |
| ENSG00000259207 | ITGB3 | 0.996835 | 1.593557 | 0.053173 | 0.088157 |
| ENSG00000138131 | LOXL4 | 0.980926 | 1.458176 | 0.076798 | 0.109177 |

As can be seen, the Merlin genes are not the highest performing genes in the dataset.

### Example 2: CP-GEP Models for Predicting Relapse

The R glmnet package (version 4.1-1) was used to fit the LASSO models. Briefly, this method fits a penalized partial likelihood estimation algorithm to a Cox model λ(t|x)=λ₀ (t) exp{Xβ}, subject to Σ|βⱼ|≤s. Conditioning on the penalty term s allows for both parameter estimation and feature selection, as variables whose parameter estimates are shrunk to 0 are effectively removed from the prediction model. The number of relapses in the study (N=95) prohibited us from splitting the study set into training and testing sets. An alternative solution to this external validation approach is to internally validate the model by repeated cross-validation and average the predictive performance across the multiple test sets. This double loop cross-validation approach was as described elsewhere (Bellomo et al., JCO Precision Oncology, 2020:319-34 (2020)). In brief, this approach (1) randomly sorts the study set; (2) uses 10-fold cross-validation to estimate the LASSO penalty term; and (3) uses 3-fold cross-validation and the penalty term from (2) to build/train a predictive model on two-thirds of the data and test the model on the remaining one-third, resulting in 3 predictive models. This process is repeated 100 times, generating a total of 300 predictive models. Due to the way the 3-fold cross-validation approach assigns subjects to training and testing sets, each subject is included in exactly 100 of the 300 model test sets. Final time-specific relapse probability estimates for a given subject are then calculated by averaging their survival estimates at various times post-diagnosis across the 100 test set models. The overall predictive capability of the model was assessed using a survival-based concordance (c-) statistic, and time-specific predictive capabilities were assessed at various years post-diagnosis using receiver operating characteristics (ROC curves). The variables most predictive of relapse were determined based on the proportion of times they were included in the 300 LASSO models. One final model was then created by fitting these most predictive variables with Cox LASSO regression using a penalty term equal to the average of the penalty terms from the 100 loops.

### Gene Filtering Approach.

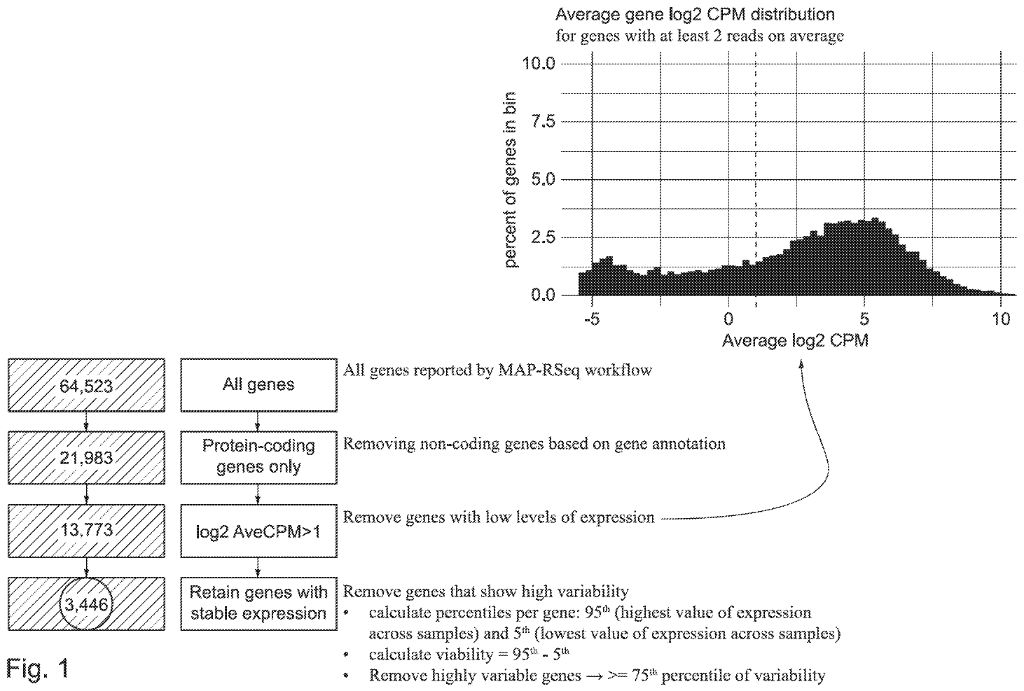

All genes reported by the MAP-RSeq workflow (N=64,523) were filtered as follows:
noncoding genes were removed.
genes with low expression were removed.
genes with low variability in expression across samples were removed.
See FIG. 1 for details.

### Models Used in LASSO

The following two models were created:
1. Clinical variables (CV)+Mutation burden value (MBV)+BRAFV600+NRASG12+NRASQ61+Variable Gene Expression (LASSO Survival model)
2. Clinical variables (CV)+Mutation burden value (MBV)+BRAFV600+NRASG12+NRASQ61+Variable Gene Expression (LASSO Logistic regression model to determine SLNb status)
   Blom inverse normal transformation was applied for variables “Breslow,” “mitotic_rate_level” and Gene expressions.
   Clinical variables (CV) includes:
      Time, Relapse status, (Only used in model 1)
      SLNb_status (Only used in model 2)
      Calc_age_primary_mel_biopsy, sex, ulceration_YES, ulceration_unknown, breslow, mitotic_rate_level
   For mutations BRAFV600, NRASG12, NRASQ61 value was set as 0 in WT and 1 if mutation was present.

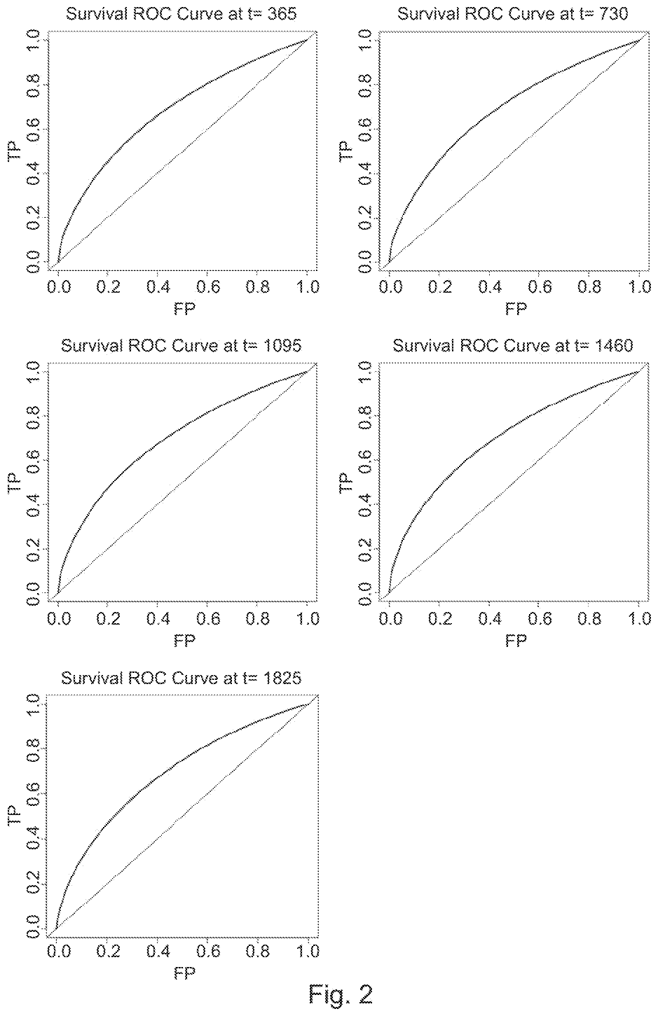

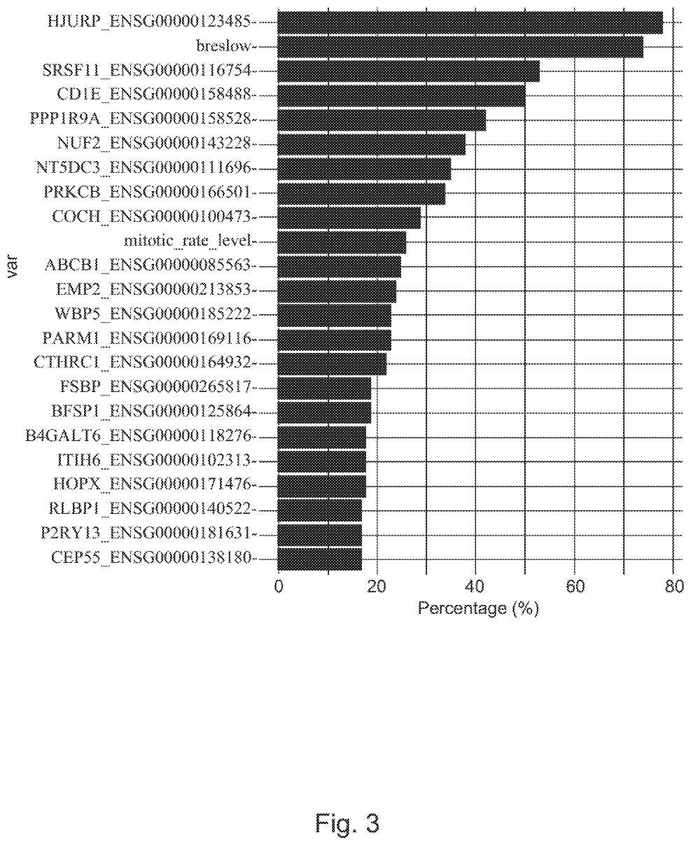

ROCs at different time points are shown in FIG. 2. Variables that appear at least 10% of times in the 300 models are shown in FIG. 3. Tumor mutational burden (TMB) did not add any significant value for predicting melanoma relapse.

The AUC for CV+gene expression+mutation burden value (MBV)+BRAFV600, NRASG12, and NRASQ61 mutations are shown in Table 2.

TABLE 2
AUC with CV + Gene expression + MBV + Mutations.

| Year | Clinical |
|---|---|
| 1 | 0.6815132 |
| 2 | 0.6853817 |
| 3 | 0.6913476 |
| 4 | 0.6979536 |
| 5 | 0.6903177 |

Genes demonstrating differential expression that can be used to predict melanoma relapse are shown in FIG. 4. A positive coefficient value for genes analyzed by this approach indicates that increased gene expression is correlated with increased patient risk while a negative coefficient value for genes indicates that decreased gene expression is correlated with increased patient risk.

Together these results demonstrate that an increased level of a HJURP polypeptide, an increased level of a NUF2 polypeptide, an increased level of a PPPIR9A polypeptide, an increased level of a SRSF11 polypeptide, an increased level of a NT5DC3 polypeptide, a decreased level of a PARM1 polypeptide, a decreased level of an EMP2 polypeptide, a decreased level of a PRKCB polypeptide, and a decreased level of a CD1E polypeptide in melanoma cells of a mammal having melanoma can be used to identify that mammal as being likely to relapse.

### Example 3: A Prognostic Gene Expression Signature for Primary Cutaneous Melanoma

### Methods:

### Patient Cohort

The study cohort was derived from a previously published cohort of 854 patients diagnosed with stage I-III malignant melanoma who had an SLNb performed within 90 days of their diagnosis (Quattrocchi et al., Int. J. Dermatol., 59:1241-8 (2020)). Briefly, patients with primary cutaneous melanoma with known SLN status were retrospectively identified by electronic searches of pathology reports. Charts were then reviewed for eligibility criteria (see next paragraph), and if met, diagnostic biopsy tissue was requested. Patients underwent SLNb. Patients who presented with stage IV disease or who developed stage IV disease within 90 days of melanoma diagnosis were excluded from the cohort.

For the purpose of this analysis, patients with stage IIB-C (N=54) or stage III disease (N=203) at diagnosis (patients with advanced primary tumors or regional metastasis) were excluded. Thus, all patients included were with stage I-IIA disease (patients with early-stage primaries and no evidence of metastasis).

All specimens were analyzed by quantitative polymerase chain reaction (qPCR). 23 additional patients were excluded for poor qPCR amplification. Thus, a total of 574 patients were included into the analysis.

### Quantitative Polymerase Chain Reaction

Quantitative polymerase chain reaction (PCR) was performed as described elsewhere (Bellomo et al., JCO Precision Oncology, 2020:319-34 (2020)). Briefly, RNA purification was from formalin-fixed paraffin-embedded tissue (QIAGEN, Hilden, Germany). Quantitative reverse transcription PCR was done using the BioMark HD System and dynamic array integrated fluid circuits (Fluidigm, South San Francisco, CA). All cDNA was pre-amplified (TaqMan PreAmp Master Mix, Applied Biosystems, Foster City, CA). Array-based quantitative PCR was developed with the help of the TaqMan Gene Expression Master Mix (Applied Biosystems). After thermal cycling, raw Ct data were checked for linear amplification. Gene expression was corrected by the mean of housekeeping genes (RLP0, RLP8, and β-actin) using the ΔCt method.

### Variable Selection

Clinical characteristics considered included age at diagnosis (years); gender; Breslow depth (mm); mitotic rate level; extent of tumor infiltrating lymphocytes (absent, non-brisk, brisk); histologic type (superficial spreading, nodular, desmoplastic, lentigo maligna, aeral lentiginous, spindled, dermal, spitzoid, nevoid, missed, other/unclassifiable); biopsy location (head/neck, trunk, upper extremities, lower extremities, aeral); and presence of ulceration, regression, microsatellitosis, and angiolymphatic invasion.

Gene expression was determined for the following 138 genes:

ABCC3, ACTGIP20, ACVR1C, ADAM12, ADAMTS19, ADIPOQ, AGRN, ALDH2, APOD, ARSG, BCL2L11, BMBPR1B, BTC, CBX3, CD1E, CD200R1, CD274, CD44, CD68, CDH11, CDH2, CDKN1A, CDKN2A, CENPF, CNTN1, COL4A1, COL6A1, CORO2B, CPN1, CSRC, CTGE, CTLA4, CTNNB1, CXCL1, CXCL9, DCD, DLC1, EGFR, ELOVL2, ERBB2, ETV7, EYA4, FCER1A, FGFR2, FLT1, FN1, GDF11, GDF15, GPRC5B, HEY1, HHATL, HJURP, HNRNPA1P33, HOPX, IGF1R, IGFBP5, IL8, INHBA, ITGA2, ITGA3, ITGA5, ITGB1, ITGB3, ITGB5, ITGB6, ITGB7, ITGB8, KCNK10, KCNQ5, KRT14, LAMA4, LAMB1, LAMB3, LGALS1, LHFPL3, LIMA1, LOXL1, LOXL3, LOXL4, LRRC15, MITE, MKI67, MLANA, MMP10, MMP14, MMP3, MOBP, MX1, MYBL2, MYH4, MYL9, NALON, NR2E1, NT5DC3, NUF2, OR52N4, PARM1, PDCD1, PIM2, PLAT, PLAU, PLOD3, PPP1R1A, PPPIR9A, PRAME, PRKCB, PTK2, RARA, RGS1, RLBP1, SDC3, SERPINE1, SERPINE2, SIRPG, SLC24A2, SMAD4, SPP1, SRSF11, SSX2B, TFAP2B, TGFB1, TGFB2, TGFB3, TGFB1, TGFBR1, TGFBR2, THBS1, THBS2, TMEM163, TMEM98, TNC, TOX, TP53, TUBA8, TUBB3, UPP1, VIM, WBP5

Missing expression values occurred at a rate of 0.3% of the total number of expression values (255/79212). Data were filled for regression analysis using mean imputation.

### Statistical Methods

The primary endpoint for the study was time to relapse after initial diagnosis of melanoma. Subjects who died, were lost to follow-up, or reached the end of the study period without relapse were censored at those respective timepoints. Gene expression was examined using normalized cycle threshold values (ΔCt values). All continuous variables were modeled assuming a log-linear association with time to relapse.

Multivariate models to predict time to relapse were built using Cox proportional hazards, least absolute shrinkage and selection operator (LASSO) regression with a double loop cross-validation (DLCV) component. The R glmnet package (version 4.1-1) was used to fit the LASSO models (Tibshirani et al., J. R. Stat. Soc., 58:267-88 (1996); and Tibshirani et al., Stat. Med., 16:385-95 (1997)). Briefly, this method fits a penalized partial likelihood estimation algorithm to a Cox model λ(t|x)=λ₀ (t) exp{Xβ}, subject to Σ|βⱼ|≤. Conditioning on the penalty term s allows for both parameter estimation and feature selection, as variables whose parameter estimates are shrunk to 0 are effectively removed from the prediction model. The number of relapses in the study (N=85) prohibited splitting the study set into training and testing sets. An alternative solution to this external validation approach is to internally validate the model by repeated cross-validation and average the predictive performance across the multiple test sets. This double loop cross-validation approach has been described elsewhere (Wessels et al. Bioinformatics 2005; 21:3755-62) and has been employed by us previously to predict sentinel lymph node metastasis in the study set (Bellomo et al., JCO Precision Oncology, 2020:319-34 (2020)). In brief, this approach (1) randomly sorts the study set; (2) uses 10-fold cross-validation to estimate the LASSO penalty term; and (3) uses 3-fold cross-validation and the penalty term from (2) to build/train a predictive model on two-thirds of the data and test the model on the remaining one-third, resulting in 3 predictive models. This process is repeated 100 times, generating a total of 300 predictive models. Due to the way the 3-fold cross-validation approach assigns subjects to training and testing sets, each subject is included in exactly 100 of the 300 model test sets. Final time-specific relapse probability estimates for a given subject are then calculated by averaging their survival estimates at various times post-diagnosis across the 100 test set models. The overall predictive capability of the model was assessed using a survival-based concordance (c-)statistic, and time-specific predictive capabilities were assessed at various years post-diagnosis using receiver operating characteristics (ROC curves). The variables most predictive of relapse were determined based on the proportion of times they were included in the 300 LASSO models. One final model was then created by fitting these most predictive variables with Cox LASSO regression using a penalty term equal to the average of the penalty terms from the 100 loops.

### Results:

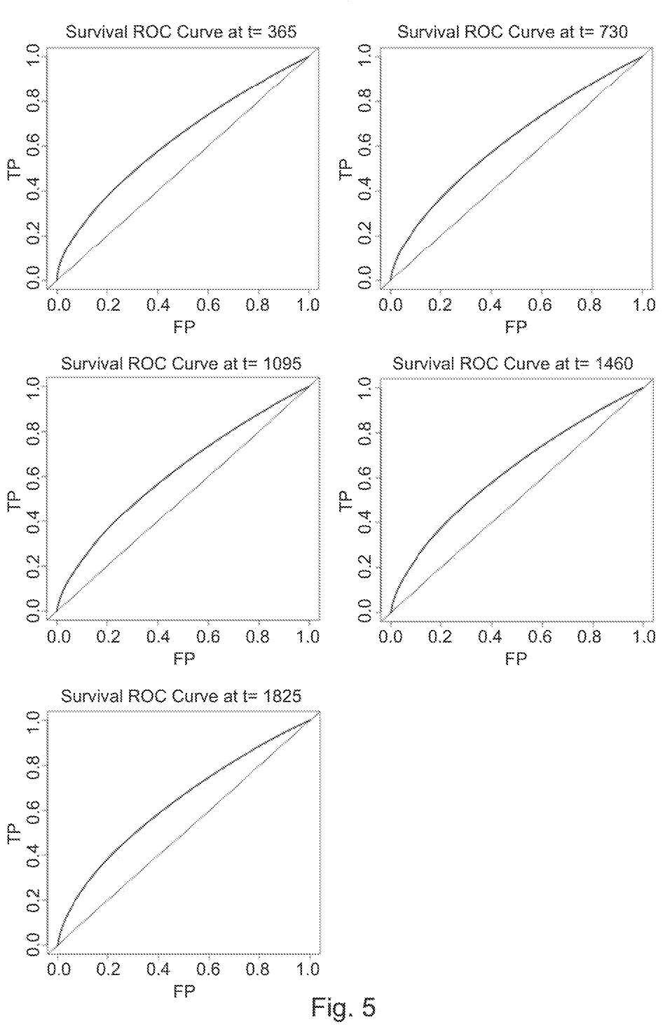

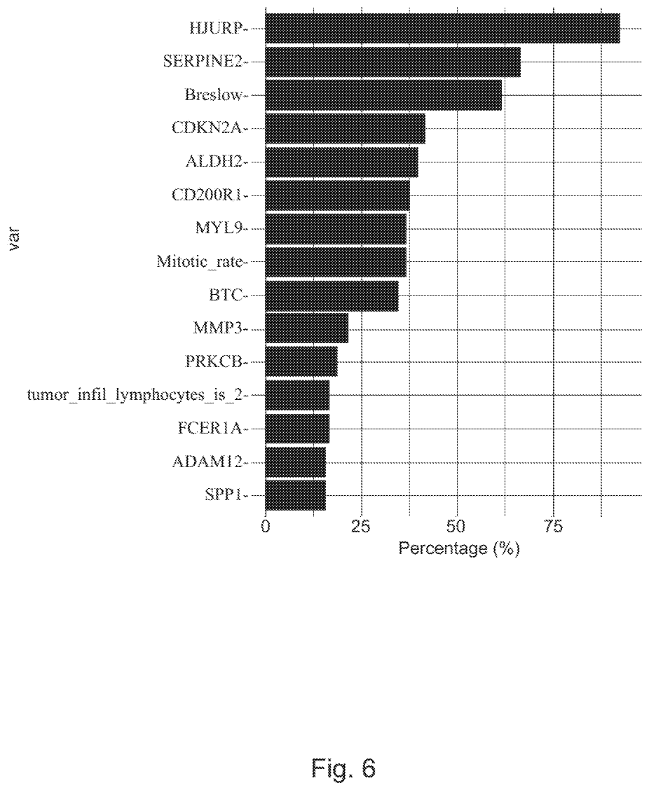

ROCs at different time points are shown in FIG. 5. Variables that appear at least 10% of times in the 300 models are shown in FIG. 6.

The AUC for CV+qPCRGene expression are shown in Table 3.

TABLE 3
AUC with CV + qPCRGene expression

| Year | Clinical |
|---|---|
| 1 | 0.6256837 |
| 2 | 0.6210832 |
| 3 | 0.6176151 |
| 4 | 0.6264475 |
| 5 | 0.6317473 |

Genes demonstrating differential expression that can be used to predict melanoma relapse are shown in FIG. 7. A positive coefficient value for genes analyzed by this approach indicates that reduced gene expression is correlated with increased patient risk while a negative coefficient value for genes indicates that increased gene expression is correlated with increased patient risk.

Together these results demonstrate that an increased level of HJURP polypeptide, an increased level of SERPINE2 polypeptide, a decreased level of CD200R1 polypeptide, and a decreased level of ALDH2 polypeptide in melanoma cells of a mammal having melanoma can be used to identify that mammal as being likely to relapse.

### Example 4: Polypeptide and mRNA Levels

Levels of a HJURP polypeptide (and mRNA encoding a HJURP polypeptide), a NUF2 polypeptide (and mRNA encoding a NUF2 polypeptide), a PPPIR9A polypeptide (and mRNA encoding a PPP1R9A polypeptide), a SRSF11 polypeptide (and mRNA encoding a SRSF11 polypeptide), a NT5DC3 polypeptide (and mRNA encoding a NT5DC3 polypeptide), a SERPINE2 polypeptide (and mRNA encoding a SERPINE2 polypeptide), a PARM1 polypeptide (and mRNA encoding a PARM1 polypeptide), an EMP2 polypeptide (and mRNA encoding an EMP2 polypeptide), a PRKCB polypeptide (and mRNA encoding a PRKCB polypeptide), a CD1E polypeptide (and mRNA encoding a CD1E polypeptide), an ALDH2 polypeptide (and mRNA encoding an ALDH2 polypeptide), and a CD200R1 polypeptide (and mRNA encoding a CD200R1 polypeptide) in humans were determined.

HJURP

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 86 | kDa |
| HJURP | | |
| HJURP cDNA length | 3157 | bp |
| Gene/Protein | HJURP | |

| | ratio: | conc | total | total | | total | total | | total | total |
|---|---|---|---|---|---|---|---|---|---|---|
| | actin/HJURP | HJURP | protein | mRNA | | protein | mRNA | | protein | mRNA |
| control melanoma | | | | | | | | | | |
| low relative | 104.8949952 | 1.36645E−12 | 1.4 pg | 0.17 pg | control | 1.4 | 0.17 | control | 100% | 100% |
| to actin | | | | | low | | | low | | |
| high relative | 61.34701368 | 2.33644E−12 | 2.3 pg | 0.3 pg | control | 2.3 | 0.3 | control | 164% | 176% |
| to actin | | | | | high | | | high | | |
| high-risk melanoma | | | | | | | | | | |
| low relative | 50.784454 | 2.82239E−12 | 2.8 pg | 0.36 pg | high risk | 2.8 | 0.36 | high risk | 200% | 212% |
| to actin | | | | | low | | | low | | |
| high relative | 29.61594485 | 4.83974E−12 | 4.8 pg | 0.62 pg | high risk | 4.8 | 0.62 | high risk | 343% | 365% |
| to actin | | | | | high | | | high | | |

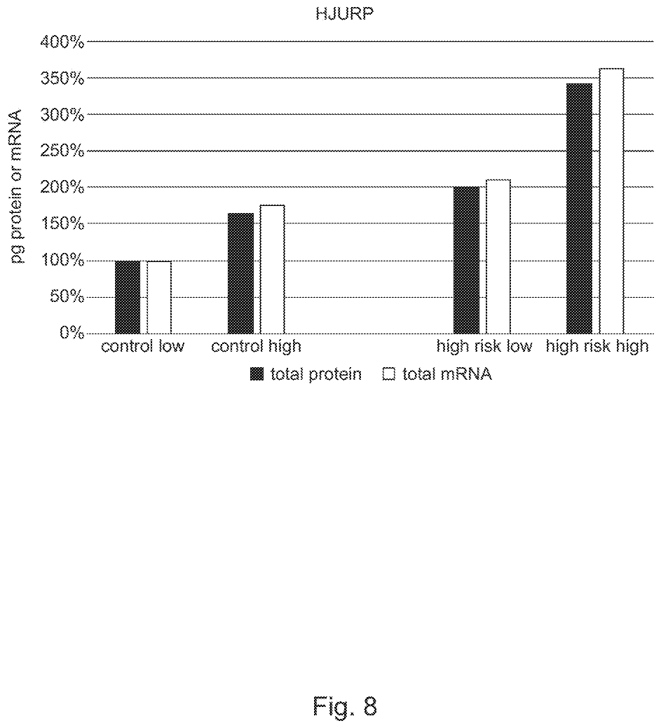

Polypeptide and mRNA levels are also shown in FIG. 8.

NUF2

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 54 | kDa |
| NUF2 | | |
| NUF2 cDNA length | 1964 | bp |
| Gene/Protein | NUF2 | |

| | ratio: | conc | total | total | | total | total | | total | total |
|---|---|---|---|---|---|---|---|---|---|---|
| | actin/NUF2 | NUF2 | protein | mRNA | | protein | mRNA | | protein | mRNA |
| control melanoma | | | | | | | | | | |
| low relative | 100.2839952 | 8.97451E−13 | 0.9 pg | 0.11 pg | control | 0.9 | 0.11 | control | 100% | 100% |
| to actin | | | | | low | | | low | | |
| high relative | 58.71899924 | 1.53272E−12 | 1.5 pg | 0.19 pg | control | 1.5 | 0.19 | control | 167% | 173% |
| to actin | | | | | high | | | high | | |
| high-risk melanoma | | | | | | | | | | |
| low relative | 51.87280432 | 1.73501E−12 | 1.7 pg | 0.22 pg | high risk | 1.7 | 0.22 | high risk | 189% | 200% |
| to actin | | | | | low | | | low | | |
| high relative | 32.71881732 | 2.75071E−12 | 2.8 pg | 0.35 pg | high risk | 2.8 | 0.35 | high risk | 311% | 318% |
| to actin | | | | | high | | | high | | |

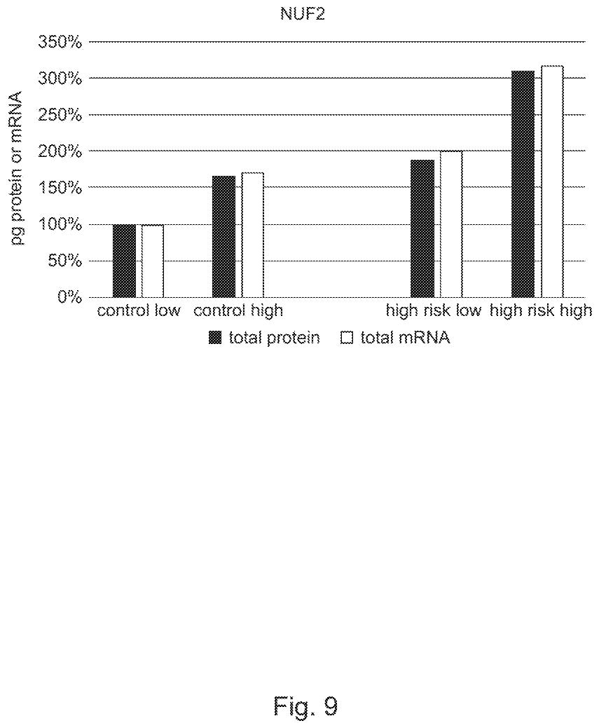

Polypeptide and mRNA levels are also shown in FIG. 9.

PPP1R9A

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 123 | kDa |
| PPP1R9A | | |
| PPP1R9A cDNA length | 10547 | bp |
| Gene/Protein | PPP1R9A | |

| | ratio: | conc | total | | total | | | total | total | | total | total |
|---|---|---|---|---|---|---|---|---|---|---|---|---|
| | actin/PPP1R9A | PPP1R9A | protein | | mRNA | | | protein | mRNA | | protein | mRNA |
| control melanoma | | | | | | | | | | | | |
| low relative | 163.505836 | 1.25378E−12 | 1.3 | pg | 0.37 | pg | control | 1.3 | 0.37 | control | 100% | 100% |
| to actin | | | | | | | low | | | low | | |
| high relative | 79.10236141 | 2.59158E−12 | 2.6 | pg | 0.77 | pg | control | 2.6 | 0.77 | control | 200% | 208% |
| to actin | | | | | | | high | | | high | | |
| high-risk melanoma | | | | | | | | | | | | |
| low relative | 68.23056118 | 3.00452E−12 | 3 | pg | 0.9 | pg | high risk | 3 | 0.9 | high risk | 231% | 243% |
| to actin | | | | | | | low | | | low | | |
| high relative | 30.75825458 | 6.66488E−12 | 6.7 | pg | 1.99 | pg | high risk | 6.7 | 1.99 | high risk | 515% | 538% |
| to actin | | | | | | | high | | | high | | |

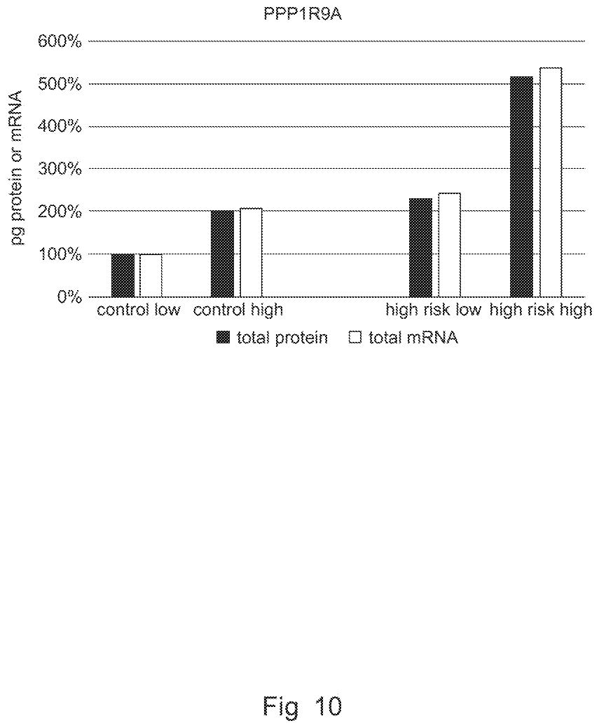

Polypeptide and mRNA levels are also shown in FIG. 10.

SRSF11

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 54 | kDa |
| SRSF11 | | |
| SRSF11 cDNA length | 3784 | bp |
| Gene/Protein | SRSF11 | |

| | ratio: | conc | total | | total | | total | total | | total | total |
|---|---|---|---|---|---|---|---|---|---|---|---|
| | actin/SRSF11 | SRSF11 | protein | | mRNA | | protein | mRNA | | protein | mRNA |
| control melanoma | | | | | | | | | | | |
| low relative | 27.91541641 | 3.22402E−12 | 3.2 | pg | 0.79 pg | control | 3.2 | 0.79 | control | 100% | 100% |
| to actin | | | | | | low | | | low | | |
| high relative | 12.80062107 | 7.03091E−12 | 7 | pg | 1.71 pg | control | 7 | 1.71 | control | 219% | 216% |
| to actin | | | | | | high | | | high | | |
| high-risk melanoma | | | | | | | | | | | |
| low relative | 13.73883662 | 6.55077E−12 | 6.6 | pg | 1.6 pg | high risk | 6.6 | 1.6 | high risk | 206% | 203% |
| to actin | | | | | | low | | | low | | |
| high relative | 6.303678236 | 1.42774E−11 | 14.3 | pg | 3.48 pg | high risk | 14.3 | 3.48 | high risk | 447% | 441% |
| to actin | | | | | | low | | | high | | |

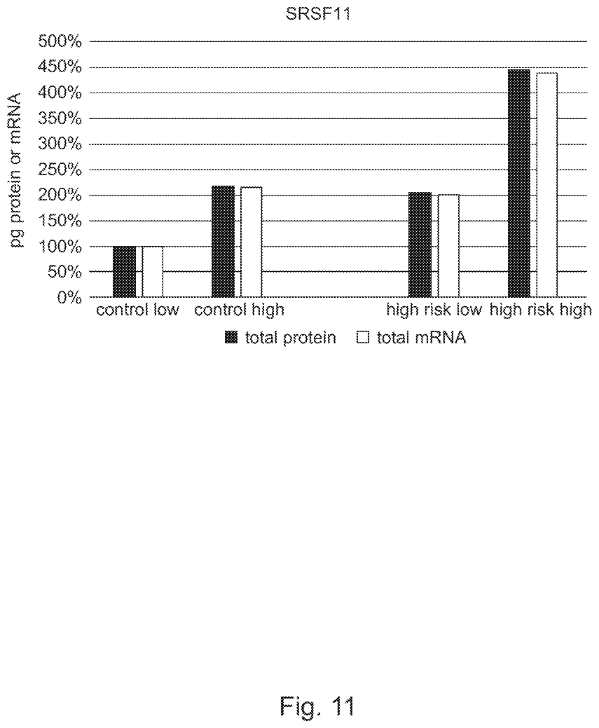

Polypeptide and mRNA levels are also shown in FIG. 11.

NT5DC3

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 63 | kDa |
| NT5DC3 | | |
| NT5DC3 cDNA length | 7244 | bp |
| Gene/Protein | NT5DC3 | |

| | ratio: | conc | total | total | | total | total | | total | total |
|---|---|---|---|---|---|---|---|---|---|---|
| | actin/NT5DC3 | NT5DC3 | protein | mRNA | | protein | mRNA | | protein | mRNA |
| control melanoma | | | | | | | | | | |
| low relative | 27.91541641 | 3.76136E−12 | 3.8 pg | 1.5 pg | control | 3.8 | 1.5 | control | 100% | 100% |
| to actin | | | | | low | | | low | | |
| high relative | 12.80062107 | 8.20273E−12 | 8.2 pg | 3.28 pg | control | 8.2 | 3.28 | control | 216% | 219% |
| to actin | | | | | high | | | high | | |
| high-risk melanoma | | | | | | | | | | |
| low relative | 13.73883662 | 7.64257E−12 | 7.6 pg | 3.06 pg | high risk | 7.6 | 3.06 | high risk | 200% | 204% |
| to actin | | | | | low | | | low | | |
| high relative | 6.303678236 | 1.66569E−11 | 16.7 pg | 6.66 pg | high risk | 16.7 | 6.66 | high risk | 439% | 444% |
| to actin | | | | | high | | | high | | |

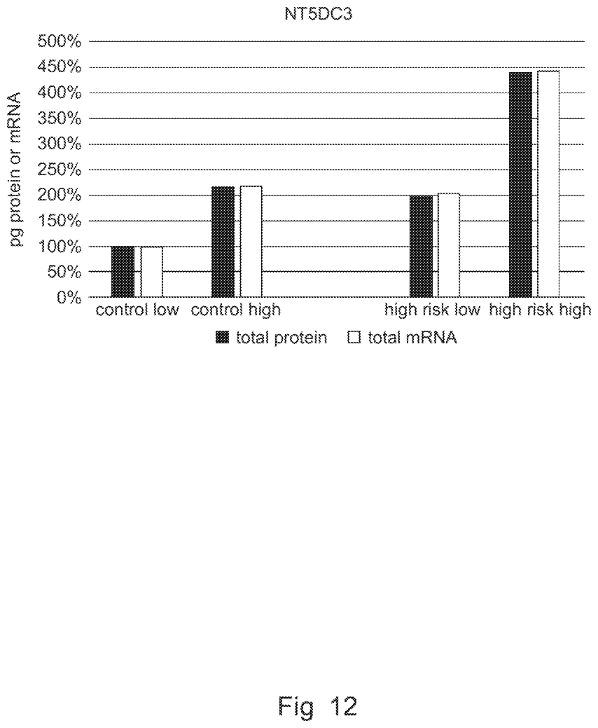

Polypeptide and mRNA levels are also shown in FIG. 12.

SERPINE2

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 44 | kDa |
| SERPINE2 | | |
| SERPINE2 cDNA length | 2569 | bp |
| Gene/Protein | SERPINE2 | |

| | ratio: | conc | total | | total | | | total | total | | total | total |
|---|---|---|---|---|---|---|---|---|---|---|---|---|
| | actin/SERPINE2 | SERPINE2 | protein | | mRNA | | | protein | mRNA | | protein | mRNA |
| control melanoma | | | | | | | | | | | | |
| low relative | 37.46519769 | 1.95737E−12 | 2 | pg | 0.4 | pg | control | 2 | 0.4 | control | 100% | 100% |
| to actin | | | | | | | low | | | low | | |
| high relative | 3.32507074 | 2.20547E−11 | 22.1 | pg | 4.48 | pg | control | 22.1 | 4.48 | control | 1105% | 1120% |
| to actin | | | | | | | high | | | high | | |
| high-risk melanoma | | | | | | | | | | | | |
| low relative | 1.158585773 | 6.32956E−11 | 63.3 | pg | 12.85 | pg | high risk | 63.3 | 12.8 | high risk | 3165% | 3200% |
| to actin | | | | | | | low | | | low | | |
| high relative | 0.167753457 | 4.37149E−10 | 437.1 | pg | 88.74 | pg | high risk | 437 | 88.7 | high risk | 21850% | 22175% |
| to actin | | | | | | | high | | | high | | |

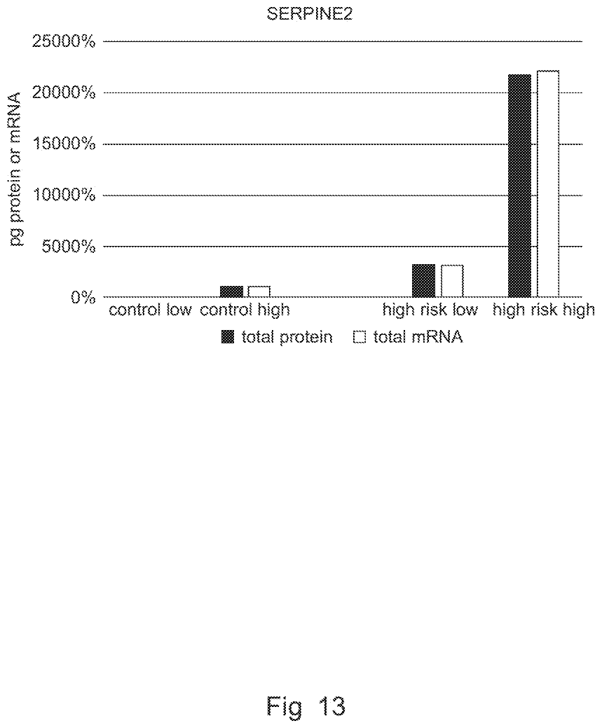

Polypeptide and mRNA levels are also shown in FIG. 13.

PARM1

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 32 | kDa |
| PARM1 | | |
| PARM1 cDNA length | 5011 | bp |
| Gene/Protein | PARM1 | |

| | ratio: | conc | total | | total | | total | total | | total | total |
|---|---|---|---|---|---|---|---|---|---|---|---|
| | actin/PARM1 | PARM1 | protein | | mRNA | | protein | mRNA | | protein | mRNA |
| control melanoma | | | | | | | | | | | |
| low relative | 31.38233101 | 1.69947E−12 | 1.7 | pg | 0.93 pg | control | 1.7 | 0.93 | control | 100% | 100% |
| to actin | | | | | | low | | | low | | |
| high relative | 17.61368264 | 3.02795E−12 | 3 | pg | 1.65 pg | control | 3 | 1.65 | control | 176% | 177% |
| to actin | | | | | | high | | | high | | |
| high-risk melanoma | | | | | | | | | | | |
| low relative | 74.32304167 | 6.98784E−13 | 0.7 | pg | 0.38 pg | high risk | 0.7 | 0.38 | high risk | 41% | 41% |
| to actin | | | | | | low | | | low | | |
| high relative | 35.69987105 | 1.49394E−12 | 1.5 | pg | 0.81 pg | high risk | 1.5 | 0.81 | high risk | 88% | 87% |
| to actin | | | | | | high | | | high | | |

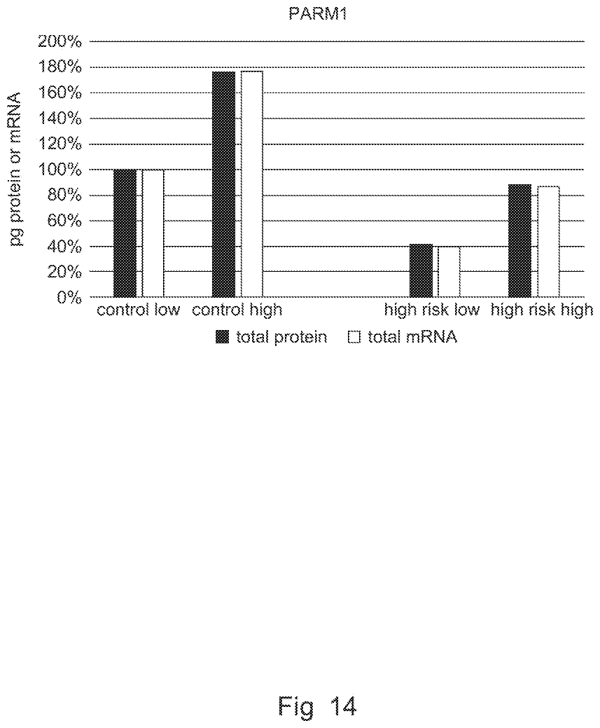

Polypeptide and mRNA levels are also shown in FIG. 14.

EMP2

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 19 | kDa |
| EMP2 | | |
| EMP2 cDNA length | 5097 | bp |
| Gene/Protein | EMP2 | |

| | ratio: | conc | total | total | | total | total | | total | total |
|---|---|---|---|---|---|---|---|---|---|---|
| | actin/EMP2 | EMP2 | protein | mRNA | | protein | mRNA | | protein | mRNA |
| control melanoma | | | | | | | | | | |
| low relative | 49.47570535 | 6.40045E−13 | 0.6 pg | 0.6 pg | control | 0.6 | 0.6 | control | 100% | 100% |
| to actin | | | | | low | | | low | | |
| high relative | 21.24 | 1.4909E−12 | 1.5 pg | 1.39 pg | control | 1.5 | 1.39 | control | 250% | 232% |
| to actin | | | | | high | | | high | | |
| high-risk melanoma | | | | | | | | | | |
| low relative | 93.7 | 3.37958E−13 | 0.3 pg | 0.32 pg | high | 0.3 | 0.32 | high | 50% | 53% |
| to actin | | | | | risk low | | | risk low | | |
| high relative | 64.13615263 | 4.93741E−13 | 0.5 pg | 0.46 pg | high | 0.5 | 0.46 | high | 83% | 77% |
| to actin | | | | | risk high | | | risk high | | |

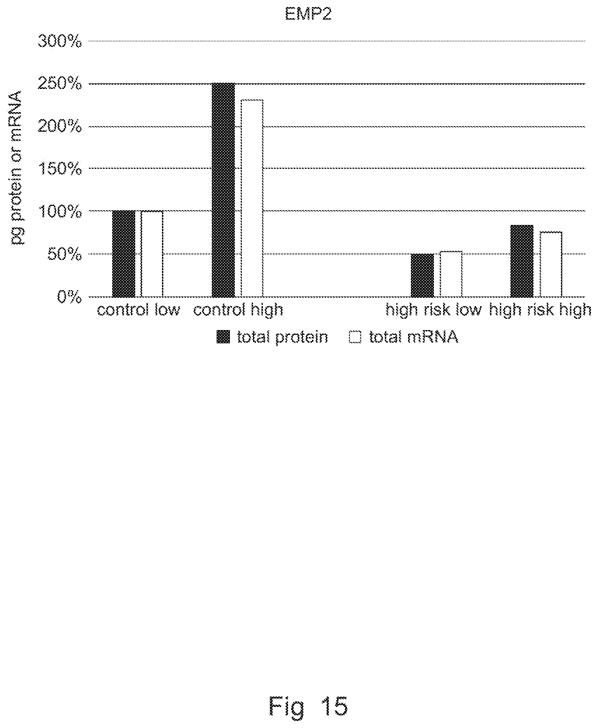

Polypeptide and mRNA levels are also shown in FIG. 15.

PRKCB

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 77 | kDa |
| PRKCB | | |
| PRKCB cDNA length | 8010 | bp |
| Gene/Protein | PRKCB | |

| | ratio: | conc | total | total | | total | total | | total | total |
|---|---|---|---|---|---|---|---|---|---|---|
| | actin/PRKCB | PRKCB | protein | mRNA | | protein | mRNA | | protein | mRNA |
| control melanoma | | | | | | | | | | |
| low relative to actin | 44.41242457 | 2.88958E−12 | 2.9 pg | 1.05 pg | control | 2.9 | 1.05 | control | 100% | 100% |
| | | | | | low | | | low | | |
| high relative to actin | 24.55610517 | 5.22613E−12 | 5.2 pg | 1.89 pg | control | 5.2 | 1.89 | control | 179% | 180% |
| | | | | | high | | | high | | |
| high-risk melanoma | | | | | | | | | | |
| low relative to actin | 91.51585193 | 1.40231E−12 | 1.4 pg | 0.51 pg | high | 1.4 | 0.51 | high | 48% | 49% |
| | | | | | risk low | | | risk low | | |
| high relative to actin | 50.08594986 | 2.56226E−12 | 2.6 pg | 0.93 pg | high | 2.6 | 0.93 | high | 90% | 89% |
| | | | | | risk high | | | risk high | | |

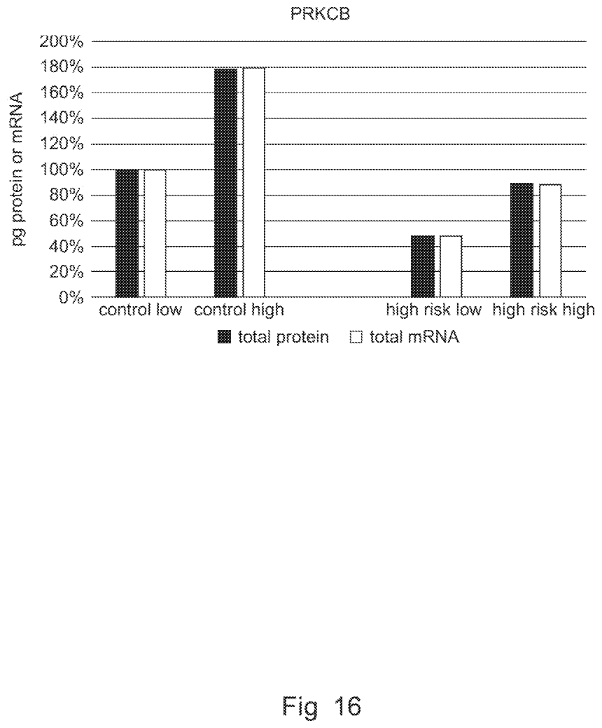

Polypeptide and mRNA levels are also shown in FIG. 16.

CD1E

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 44 | kDa |
| CD1E | | |
| CD1E cDNA length | 1919 | bp |
| Gene/Protein | CD1E | |

| | ratio: | conc | total | total | | total | total | | total | total |
|---|---|---|---|---|---|---|---|---|---|---|
| | actin/CD1E | CD1E | protein | mRNA | | protein | mRNA | | protein | mRNA |
| control melanoma | | | | | | | | | | |
| low relative | 12.42891409 | 5.90022E−12 | 5.9 pg | 0.89 pg | control | 5.9 | 0.89 | control | 100% | 100% |
| to actin | | | | | low | | | low | | |
| high relative | 9.514064713 | 7.70789E−12 | 7.7 pg | 1.17 pg | control | 7.7 | 1.17 | control | 131% | 131% |
| to actin | | | | | high | | | high | | |
| high-risk melanoma | | | | | | | | | | |
| low relative | 17.38681034 | 4.21776E−12 | 4.2 pg | 0.64 pg | high | 4.2 | 0.64 | high | 71% | 72% |
| to actin | | | | | risk low | | | risk low | | |
| high relative | 13.28003537 | 5.52207E−12 | 5.5 pg | 0.84 pg | high | 5.5 | 0.84 | high | 93% | 94% |
| to actin | | | | | risk high | | | risk high | | |

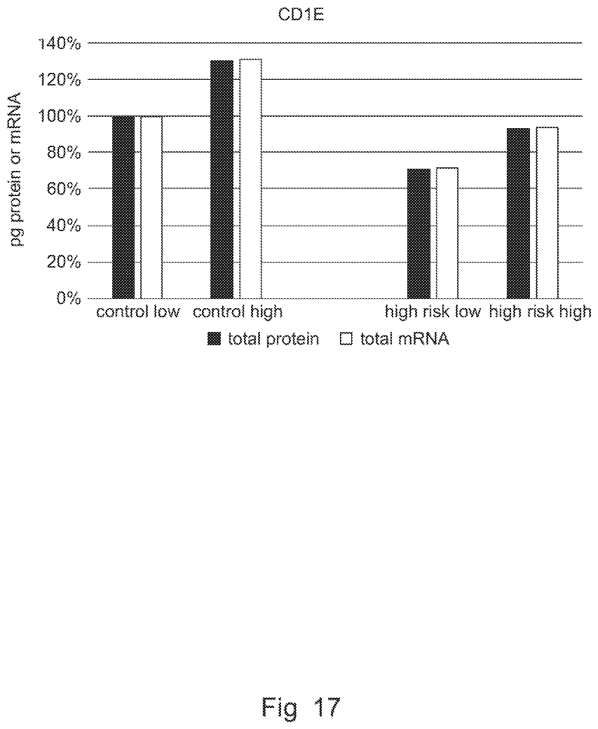

Polypeptide and mRNA levels are also shown in FIG. 17.

ALDH2

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 56 | kDa |
| ALDH2 | | |
| ALDH2 cDNA length | 9561 | bp |
| Gene/Protein | ALDH2 | |

| | ratio: | conc | total | total | | total | total | | total | total |
|---|---|---|---|---|---|---|---|---|---|---|
| | actin/ALDH2 | ALDH2 | protein | mRNA | | protein | mRNA | | protein | mRNA |
| control melanoma | | | | | | | | | | |
| low relative | 19.31137675 | 4.83308E−12 | 4.8 pg | 2.87 pg | control | 4.8 | 2.87 | control | 100% | 100% |
| to actin | | | | | low | | | low | | |
| high relative | 8.197359169 | 1.13858E−11 | 11.4 pg | 6.76 pg | control | 11.4 | 6.76 | control | 238% | 236% |
| to actin | | | | | high | | | high | | |
| high-risk melanoma | | | | | | | | | | |
| low relative | 64.75583926 | 1.44131E−12 | 1.4 pg | 0.86 pg | high | 1.4 | 0.86 | high | 29% | 30% |
| to actin | | | | | risk low | | | risk low | | |
| high relative | 25.36481079 | 3.67964E−12 | 3.7 pg | 2.18 pg | high | 3.7 | 2.18 | high | 77% | 76% |
| to actin | | | | | risk high | | | risk high | | |

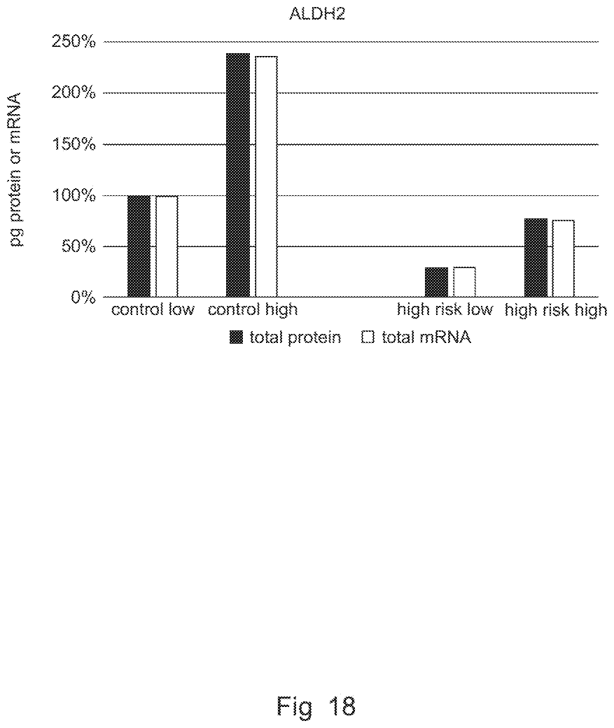

Polypeptide and mRNA levels are also shown in FIG. 18.

CD200R1

| | | |
|---|---|---|
| Total actin | 7E−11 | gram |
| protein per cell | | |
| actin molecular | 42 | kDa |
| weight protein | | |
| actin cDNA length | 1812 | bp |
| Total actin mRNA | 10.5 | pg |
| per cell | | |
| molecular weight | 39 | kDa |
| CD200R1 | | |
| CD200R1 cDNA length | 3765 | bp |
| Gene/Protein | CD200R1 | |

| | ratio: | conc | total | | total | | total | total | | total | total |
|---|---|---|---|---|---|---|---|---|---|---|---|
| | actin/CD200R1 | CD200R1 | protein | | mRNA | | protein | mRNA | | protein | mRNA |
| control melanoma | | | | | | | | | | | |
| low relative | 60.00347153 | 1.08327E−12 | 1.1 | pg | 0.36 pg | control | 1.1 | 0.36 | control | 100% | 100% |
| to actin | | | | | | low | | | low | | |
| high relative | 36.80803848 | 1.76592E−12 | 1.8 | pg | 0.59 pg | control | 1.8 | 0.59 | control | 164% | 164% |
| to actin | | | | | | high | | | high | | |
| high-risk melanoma | | | | | | | | | | | |
| low relative | 128.7174298 | 5.04982E−13 | 0.5 | pg | 0.17 pg | high | 0.5 | 0.17 | high | 45% | 47% |
| to actin | | | | | | risk low | | | risk low | | |
| high relative | 67.13698457 | 9.6817E−13 | 1 | pg | 0.32 pg | high | 1 | 0.32 | high | 91% | 89% |
| to actin | | | | | | risk high | | | risk high | | |

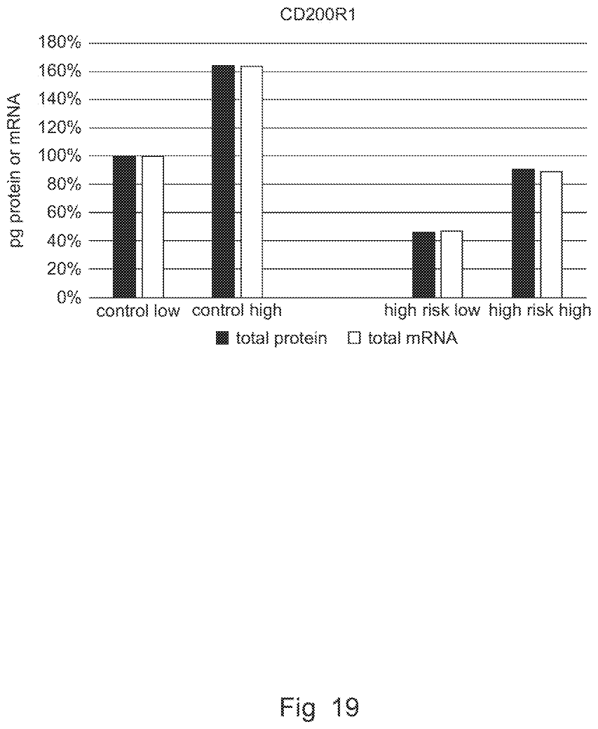

Polypeptide and mRNA levels are also shown in FIG. 19.

### Example 5: Treating PCM

A tissue sample containing one or more melanoma cells is obtained from a human having PCM. The obtained sample is examined for the presence or absence of an altered level of a HJURP polypeptide, a NUF2 polypeptide, a PPPIR9A polypeptide, a SRSF11 polypeptide, a NT5DC3 polypeptide, a SERPINE2 polypeptide, a PARM1 polypeptide, an EMP2 polypeptide, a PRKCB polypeptide, a CD1E polypeptide, an ALDH2 polypeptide, a CD200R polypeptide.

If the presence of at least 4 of: an increased level of a HJURP polypeptide, an increased level of a NUF2 polypeptide, an increased level of a PPP1R9A polypeptide, an increased level of a SRSF11 polypeptide, an increased level of a NT5DC3 polypeptide, a decreased level of a PARM1 polypeptide, a decreased level of an EMP2 polypeptide, a decreased level of a PRKCB polypeptide, a decreased level of a CD1E polypeptide, a decreased level of an ALDH2 polypeptide, and a decreased level of a CD200R polypeptide are detected in the sample, then the PCM is identified as being likely to relapse (e.g., likely to relapse following treatment), and the human is subjected to surgery to remove the PCM and is administered one or more adjuvant therapies.

The surgery in combination with the one or more adjuvant therapies can reduce number of cancer cells within the human while minimizing the risk of relapse.

### Example 6: Treating PCM

A tissue sample containing one or more melanoma cells is obtained from a human having PCM. The obtained sample is examined for the presence or absence of an altered level of a HJURP polypeptide, a NUF2 polypeptide, a PPPIR9A polypeptide, a SRSF11 polypeptide, a NT5DC3 polypeptide, a SERPINE2 polypeptide, a PARM1 polypeptide, an EMP2 polypeptide, a PRKCB polypeptide, a CD1E polypeptide, an ALDH2 polypeptide, a CD200R polypeptide.

If the absence of an increased level of a HJURP polypeptide, an increased level of a NUF2 polypeptide, an increased level of a PPPIR9A polypeptide, an increased level of a SRSF11 polypeptide, an increased level of a NT5DC3 polypeptide, a decreased level of a PARM1 polypeptide, a decreased level of an EMP2 polypeptide, a decreased level of a PRKCB polypeptide, a decreased level of a CD1E polypeptide, a decreased level of an ALDH2 polypeptide, and a decreased level of a CD200R polypeptide is detected in the sample, then the PCM is identified as not being likely to relapse (e.g., likely to relapse following treatment), and the human is subjected to surgery to remove the PCM without any adjuvant therapy.

The surgery can reduce number of cancer cells within the human while sparing the human from unnecessary toxicities and/or expenses of adjuvant therapies.

### Example 7: Treating PCM

A tissue sample containing one or more melanoma cells is obtained from a human having PCM. The obtained sample is examined for the level of a nucleic acid (e.g., an mRNA) encoding a HJURP polypeptide, the level of a nucleic acid (e.g., an mRNA) encoding a NUF2 polypeptide, the level of a nucleic acid (e.g., an mRNA) encoding a PPPIR9A polypeptide, the level of a nucleic acid (e.g., an mRNA) encoding a SRSF11 polypeptide, the level of a nucleic acid (e.g., an mRNA) encoding a NT5DC3 polypeptide, the level of a nucleic acid (e.g., an mRNA) encoding a SERPINE2 polypeptide, the level of a nucleic acid (e.g., an mRNA) encoding a PARM1 polypeptide, the level of a nucleic acid (e.g., an mRNA) encoding an EMP2 polypeptide, the level of a nucleic acid (e.g., an mRNA) encoding a PRKCB polypeptide, the level of a nucleic acid (e.g., an mRNA) encoding a CD1E polypeptide, the level of a nucleic acid (e.g., an mRNA) encoding an ALDH2 polypeptide, and the level of a nucleic acid (e.g., an mRNA) encoding a CD200R polypeptide.

If at least 4 of: the level of a nucleic acid (e.g., an mRNA) encoding a HJURP polypeptide is greater than 0.3 pg/melanoma cell, the level of a nucleic acid (e.g., an mRNA) encoding a NUF2 polypeptide is greater than 0.19 pg/melanoma cell, the level of a nucleic acid (e.g., an mRNA) encoding a PPPIR9A polypeptide is greater than 0.77 pg/melanoma cell, the level of a nucleic acid (e.g., an mRNA) encoding a SRSF11 polypeptide is greater than 1.71 pg/melanoma cell, the level of a nucleic acid (e.g., an mRNA) encoding a NT5DC3 polypeptide is greater than 3.28 pg/melanoma cell, the level of a nucleic acid (e.g., an mRNA) encoding a SERPINE2 polypeptide is greater than 4.48 pg/melanoma cell, the level of a nucleic acid (e.g., an mRNA) encoding a PARM1 polypeptide is less than 0.93 pg/melanoma cell, the level of a nucleic acid (e.g., an mRNA) encoding an EMP2 polypeptide is less than 0.6 pg/melanoma cell, the level of a nucleic acid (e.g., an mRNA) encoding a PRKCB polypeptide is less than 1.05 pg/melanoma cell, the level of a nucleic acid (e.g., an mRNA) encoding a CD1E polypeptide is less than 0.89 pg/melanoma cell, the level of a nucleic acid (e.g., an mRNA) encoding an ALDH2 polypeptide is less than 2.87 pg/melanoma cell, and the level of a nucleic acid (e.g., an mRNA) encoding a CD200R polypeptide is less than 0.36 pg/melanoma cell, then the PCM is identified as being likely to relapse (e.g., likely to relapse following treatment), and the human is subjected to surgery to remove the PCM and is administered one or more adjuvant therapies.

The surgery in combination with the one or more adjuvant therapies can reduce number of cancer cells within the human while minimizing the risk of relapse.

### Example 8: Treating PCM

A tissue sample containing one or more melanoma cells is obtained from a human having PCM. The obtained sample is examined for the level of a nucleic acid (e.g., an mRNA) encoding a HJURP polypeptide, the level of a nucleic acid (e.g., an mRNA) encoding a NUF2 polypeptide, the level of a nucleic acid (e.g., an mRNA) encoding a PPPIR9A polypeptide, the level of a nucleic acid (e.g., an mRNA) encoding a SRSF11 polypeptide, the level of a nucleic acid (e.g., an mRNA) encoding a NT5DC3 polypeptide, the level of a nucleic acid (e.g., an mRNA) encoding a SERPINE2 polypeptide, the level of a nucleic acid (e.g., an mRNA) encoding a PARM1 polypeptide, the level of a nucleic acid (e.g., an mRNA) encoding an EMP2 polypeptide, the level of a nucleic acid (e.g., an mRNA) encoding a PRKCB polypeptide, the level of a nucleic acid (e.g., an mRNA) encoding a CD1E polypeptide, the level of a nucleic acid (e.g., an mRNA) encoding an ALDH2 polypeptide, and the level of a nucleic acid (e.g., an mRNA) encoding a CD200R polypeptide.

If the level of a nucleic acid (e.g., an mRNA) encoding a HJURP polypeptide is from about 0.17 pg/melanoma cell to about 0.3 pg/melanoma cell, the level of a nucleic acid (e.g., an mRNA) encoding a NUF2 polypeptide is from about 0.11 pg/melanoma cell to about 0.19 pg/melanoma cell, the level of a nucleic acid (e.g., an mRNA) encoding a PPPIR9A polypeptide is from about 0.37 pg/melanoma cell to about 0.77 pg/melanoma cell, the level of a nucleic acid (e.g., an mRNA) encoding a SRSF11 polypeptide is from about 0.79 pg/melanoma cell to about 1.71 pg/melanoma cell, the level of a nucleic acid (e.g., an mRNA) encoding a NT5DC3 polypeptide is from about 1.5 pg/melanoma cell to about 3.28 pg/melanoma cell, the level of a nucleic acid (e.g., an mRNA) encoding a SERPINE2 polypeptide is from about 0.4 pg/melanoma cell to about 4.48 pg/melanoma cell, the level of a nucleic acid (e.g., an mRNA) encoding a PARM1 polypeptide is from about 0.93 pg/melanoma cell to about 1.65 pg/melanoma cell, the level of a nucleic acid (e.g., an mRNA) encoding an EMP2 polypeptide is from about 0.6 pg/melanoma cell to about 1.39 pg/melanoma cell, the level of a nucleic acid (e.g., an mRNA) encoding a PRKCB polypeptide is from about 1.05 pg/melanoma cell to about 1.89 pg/melanoma cell, the level of a nucleic acid (e.g., an mRNA) encoding a CD1E polypeptide is from about 0.89 pg/melanoma cell to about 1.17 pg/melanoma cell, the level of a nucleic acid (e.g., an mRNA) encoding an ALDH2 polypeptide is from about 2.87 pg/melanoma cell to about 6.76 pg/melanoma cell, and the level of a nucleic acid (e.g., an mRNA) encoding a CD200R polypeptide is from about 0.36 pg/melanoma cell to about 0.59 pg/melanoma cell, then the PCM is identified as not being likely to relapse (e.g., likely to relapse following treatment), and the human is subjected to surgery to remove the PCM without any adjuvant therapy.

The surgery can reduce number of cancer cells within the human while sparing the human from unnecessary toxicities and/or expenses of adjuvant therapies.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for assessing a mammal having a primary cutaneous melanoma (PCM), wherein the method comprises:
(a) determining if a sample from the mammal contains the presence or absence of (1) an increased level of a holliday junction recognition protein (HJURP) polypeptide or mRNA encoding the HJURP polypeptide, (2) an increased level of a NUF2 component of NDC80 kinetochore complex (NUF2) polypeptide or mRNA encoding the NUF2 polypeptide, (3) an increased level of a protein phosphatase 1 regulatory subunit 9A (PPP1R9A) polypeptide or mRNA encoding the PPPIR9A polypeptide, (4) an increased level of a serine/arginine-rich splicing factor 11 (SRSF11) polypeptide or mRNA encoding the SRSF11 polypeptide, (5) an increased level of a 5′-nucleotidase domain-containing protein 3 (NT5DC3) polypeptide or mRNA encoding the NT5DC3 polypeptide, (6) an increased level of a serpin family E member 2 (SERPINE2) polypeptide or mRNA encoding the SERPINE2 polypeptide, (7) a decreased level of a prostate androgen-regulated mucin-like protein 1 (PARM1) polypeptide or mRNA encoding the PARM1 polypeptide, (8) a decreased level of an epithelial membrane protein 2 (EMP2) polypeptide or mRNA encoding the EMP2 polypeptide, (9) a decreased level of a protein kinase C beta type (PRKCB) polypeptide or mRNA encoding the PRKCB polypeptide, (10) a decreased level of a T-cell surface glycoprotein CD1e (CD1E) polypeptide or mRNA encoding the CD1E polypeptide, (11) a decreased level of an alcohol dehydrogenase 2 family member (ALDH2) polypeptide or mRNA encoding the ALDH2 polypeptide, or (12) a decreased level of a CD200 receptor 1 (CD200R1) polypeptide or mRNA encoding the CD200R1 polypeptide, wherein the sample is a tissue sample comprising a melanoma cell;
(b) classifying the PCM as being likely to relapse if the presence of at least 4 of (1)-(12) is determined; and
(c) classifying the PCM as not being likely to relapse if the absence of each of (1)-(12) is determined.

2.-19. (canceled)

20. A method for selecting a treatment for a mammal having a PCM primary cutaneous melanoma (PCM), wherein the method comprises:
(a1) determining that a sample from the mammal contains the presence of at least 4 of: (1) an increased level of a HJURP polypeptide or mRNA encoding the HJURP polypeptide, (2) an increased level of a NUF2 polypeptide or mRNA encoding the NUF2 polypeptide, (3) an increased level of a PPP1R9A polypeptide or mRNA encoding the PPP1R9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide, wherein the sample is a tissue sample comprising a melanoma cell; and (b1) selecting the mammal for surgery to remove the PCM and to receive an adjuvant therapy or
(a2) determining that a sample from the mammal contains the absence of: (1) an increased level of a HJURP polypeptide or mRNA encoding the HJURP polypeptide, (2) an increased level of a NUF2 polypeptide or mRNA encoding the NUF2 polypeptide, (3) an increased level of a PPP1R9A polypeptide or mRNA encoding the PPP1R9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide, wherein the sample is a tissue sample comprising a melanoma cell; and (b2) selecting the mammal for surgery to remove the PCM in the absence of an adjuvant therapy.

21. The method of claim 20, the method further comprising determining that the PCM has a Breslow depth of from about 0.8 mm to about 10 mm.

22.-23. (canceled)

24. The method of claim 20, wherein the mammal is a human.

25. The method of claim 20, wherein the adjuvant therapy comprises radiation therapy.

26. The method of claim 20, wherein the adjuvant therapy comprises administering an anti-cancer drug to the mammal.

27. The method of claim 26, wherein the anti-cancer drug is selected from the group consisting of nivolumab, ipilimumab, pembrolizumab, talimogene laherparepvec, vemurafenib, dabrafenib, encorafenib, trametinib, cobimetinib, binimetinib, atezolizumab, avelumab, and durvalumab.

28. (canceled)

29. A method for treating a mammal having primary cutaneous melanoma (PCM), wherein the method comprises subjecting the mammal to a surgery to remove the PCM and administering an adjuvant therapy to the mammal, wherein the mammal is identified as having at least 4 of: (1) an increased level of a HJURP polypeptide or mRNA encoding the HJURP polypeptide, (2) an increased level of a NUF2 polypeptide or mRNA encoding the NUF2 polypeptide, (3) an increased level of a PPP1R9A polypeptide or mRNA encoding the PPP1R9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide in a sample obtained from the mammal, wherein the sample is a tissue sample comprising a melanoma cell.

30. The method of claim 29, the method further comprising determining that the PCM has a Breslow depth of from about 0.8 mm to about 10 mm.

31. The method of claim 29, wherein the mammal is a human.

32. The method of claim 29, wherein the adjuvant therapy comprises radiation therapy.

33. The method of claim 29, wherein the adjuvant therapy comprises administering an anti-cancer drug to the mammal.

34. The method of claim 33, wherein the anti-cancer drug is selected from the group consisting of nivolumab, ipilimumab, pembrolizumab, talimogene laherparepvec, vemurafenib, dabrafenib, encorafenib, trametinib, cobimetinib, binimetinib, atezolizumab, avelumab, and durvalumab.

35. (canceled)

36. A method for treating a mammal having primary cutaneous melanoma (PCM), wherein the method comprises subjecting the mammal to a surgery to remove the PCM in the absence of an adjuvant therapy, wherein the mammal is identified as lacking: (1) an increased level of a HJURP polypeptide or mRNA encoding the HJURP polypeptide, (2) an increased level of a NUF2 polypeptide or mRNA encoding the NUF2 polypeptide, (3) an increased level of a PPP1R9A polypeptide or mRNA encoding the PPP1R9A polypeptide, (4) an increased level of a SRSF11 polypeptide or mRNA encoding the SRSF11 polypeptide, (5) an increased level of a NT5DC3 polypeptide or mRNA encoding the NT5DC3 polypeptide, (6) an increased level of a SERPINE2 polypeptide or mRNA encoding the SERPINE2 polypeptide, (7), a decreased level of a PARM1 polypeptide or mRNA encoding the PARM1 polypeptide, (8) a decreased level of an EMP2 polypeptide or mRNA encoding the EMP2 polypeptide, (9) a decreased level of a PRKCB polypeptide or mRNA encoding the PRKCB polypeptide, (10) a decreased level of a CD1E polypeptide or mRNA encoding the CD1E polypeptide, (11) a decreased level of an ALDH2 polypeptide or mRNA encoding the ALDH2 polypeptide, and (12) a decreased level of a CD200R1 polypeptide or mRNA encoding the CD200R1 polypeptide in a sample obtained from the mammal, wherein the sample is a tissue sample comprising a melanoma cell.

37. The method of claim 36, the method further comprising determining that the PCM has a Breslow depth of from about 0.1 mm to about 0.8 mm.

38. The method of claim 36, wherein the mammal is a human.

39. The method of claim 36, wherein the adjuvant therapy comprises radiation therapy.

40. The method of claim 36, wherein the adjuvant therapy comprises administering an anti-cancer drug to the mammal.

41. The method of claim 40, wherein the anti-cancer drug is selected from the group consisting of nivolumab, ipilimumab, pembrolizumab, talimogene laherparepvec, vemurafenib, dabrafenib, encorafenib, trametinib, cobimetinib, binimetinib, atezolizumab, avelumab, and durvalumab.
